(19) United States
(12) Patent Application Publication
(10) Pub. No.: US 2008/0096950 A1
(43) Pub. Date: Apr. 24, 2008

(54) Compounds Useful In Therapy

(76) Inventors: Karl Richard Gibson, Sandwich (GB); Sarah Elizabeth Skerratt, Sandwich (GB); Kevin Neil Dack, Sandwich (GB)

Correspondence Address: PFIZER INC., PATENT DEPARTMENT, MS8260-1611, EASTERN POINT ROAD, GROTON, CT, 06340, US

(21) Appl. No.: 11/866,565

(22) Filed: Oct. 3, 2007

Related U.S. Application Data
(60) Provisional application No. 60/915,471, filed on May 2, 2007.
(60) Provisional application No. 60/862,133, filed on Oct. 19, 2006.

(51) Int. Cl. A61K 31/4155; A61P 43/00; C07D 231/18

(52) U.S. Cl. 514/407; 548/365.7

(57) ABSTRACT

Compounds of Formula (I):

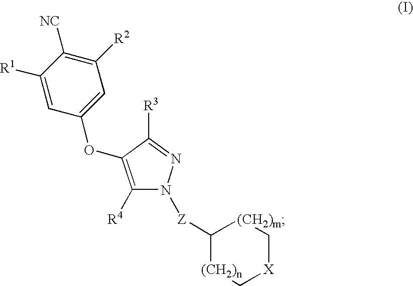

and pharmaceutically acceptable salts, solvates (including hydrates) of said compounds and salts, or prodrugs of said compound, or pharmaceutically acceptable salts or solvates of said prodrugs, wherein the substituents are as herein defined, are useful in therapy, for example they may be useful for treating progesterone-mediated conditions such as endometriosis, uterine fibroids (leiomyomata), menorrhagia, adenomyosis, primary and secondary dysmenorrhoea (including symptoms of dyspareunia, dyschexia and chronic pelvic pain), or chronic pelvic pain syndrome.

This application claims priority to U.S. Provisional Application Ser. Nos. 60/915,471, filed May 2, 2007 and 60/862,133, filed Oct. 19, 2006.

### BACKGROUND OF THE INVENTION

This invention relates to novel 4-cyanophenyloxypyrazole compounds, and their derivatives, which are useful in therapy and to processes for their preparation. It also relates to intermediates used in the preparation of such compounds and derivatives, compositions containing them and their uses, for example their use in medicine. A preferred use of the compounds is in the treatment of conditions alleviated by use of a progesterone receptor antagonist. Preferably the compounds are useful in contraception, and the treatment of endometriosis, uterine fibroids and related conditions.

Endometriosis is a common gynaecological disease that affects 10-20% women of reproductive age and manifests itself in the presence of functional ectopic endometrial glands and stroma at locations outside the uterine cavity {Prentice, A. (2001). BMJ, 323, 93-95.}. Patients with endometriosis may present with many different symptoms and severity. Most commonly this is dysmenorrhoea, but chronic pelvic pain, dyspareunia, dyschexia, menorrhagia, lower abdominal or back pain, infertility, bloating and pain on micturition are also part of the constellation of symptoms of endometriosis.

Originally described by Von Rokitansky in 1860 {Von Rokitansky, C. (1860). Ztsch. K K Gesell. der Aerzte zu Wien 37, 577-581.}, the exact pathogenesis of endometriosis is unclear {Witz, C. A. (1999). Clin. Obstet. & Gyn., 42, 566-585.; Witz, C. A. (2002). Gyn. & Obstet. Invest. 53, 52-62}, but the most widely accepted theory is the implantation, or Sampson, theory {Sampson, J. A. (1927). Am. J. Obstet. & Gyn., 14, 422-429}. The Sampson Theory proposes that the development of endometriosis is a consequence of retrograde dissemination and implantation of endometrial tissue into the peritoneal cavity during menstruation. Following attachment, the fragments of endometrium recruit a vascular supply and undergo cycles of proliferation and shedding under local and systemic hormonal controls. In women with patent fallopian tubes, retrograde menstruation appears to be a universal phenomenon {Liu, D. T. (Hitchcock, A.). Brit. J. Obstet. & Gyn., 93, 859-862.}. The disease often manifests itself as rectovaginal endometriosis or adenomyosis, ovarian cystic endometriomas and, most commonly, peritoneal endometriosis. The major sites of attachment and lesion growth within the pelvis are the ovaries, broad and round ligaments, fallopian tubes, cervix, vagina, peritoneum and the pouch of Douglas. At its most severe, endometriosis can cause profound structural modification to the peritoneal cavity, including multi-organ adhesions and fibrosis.

Symptomatic endometriosis can be managed medically and surgically, where the intention is to remove the ectopic lesion tissue. Surgical intervention can be either conservative, aiming to preserve the reproductive potential of the patient, or comparatively radical for severe disease, involving dissection of the urinary tract, bowel, and rectovaginal septum, or total abdominal hysterectomy and bilateral salpingo-oopherectomy. Medical pharmacological treatments such as the androgenic therapies, danazol and gestrinone, the constellation of GnRH agonists, buserelin, goserelin, leuprolide, nafarelin and triptorelin, GnRH antagonists, cetrorelix and abarelix, as well as the progestogens, including medroxyprogesterone acetate, induce lesion atrophy by suppressing the production of estrogen. These approaches are not without unwanted side effects; danazol and gestrinone include weight gain, hirsuitism, acne, mood changes and metabolic effects on the cardiovascular system. The group of GnRH agonists and antagonists are found to cause a profound suppression of estrogen leading to vasomotor effects (hot flashes) and depletion of bone mineral density, which restricts their use to only six months of therapy. The group of progestogens, including medroxyprogesterone acetate, suppress the gonadotropins, but do not down-regulate ovarian estrogen production to the same extent as the GnRH analogues. The side effects include irregular bleeding, bloating, weight gain and metabolic effects on the cardiovascular system.

Uterine leiomyomas {Flake, G. P., et al. (2003). Environ. Health Perspect., 111, 1037-1054.; Walker, C. L. (2002). Recent Progress in Hormone Res., 57, 277-294.}, or fibroids, are the most common benign tumours found in women and occur in the majority of women by the time they reach the menopause. Although uterine fibroids are the most frequent indication for hysterectomy in the United States, as with endometriosis, remarkably little is known about the underlying pathophysiology of the disease. As with endometriotic lesions, the presence of enlarged uterine fibroids is associated with abnormal uterine bleeding, dysmenorrhoea, pelvic pain and infertility. Outside of surgical management, medical treatments commonly used for endometriosis, such as GnRH analogues or danazol, have been shown to suppress fibroid growth by inducing a reversible hypoestrogenic state {Chrisp, P., and Goa, K. L. (1990). Drugs, 39, 523-551.; Chrisp, P., and Goa, K. L. (1991). Drugs, 41, 254-288.; De Leo, V., et al. (2002). Drug Safety 25, 759-779.; Ishihara, H., et al. (2003). Fertil. & Steril. 79, 735-742.}. However, the future disease management of both uterine fibroids and endometriosis will rely on the development of more effective, well-tolerated and safer agents than those that are currently available. Steroidal progestins (i.e., progesterone receptor agonists) are commonly used in women's health, such as in contraception and hormone therapy and for the treatment of gynecological disorders. Recent studies in women and in nonhuman primates also indicate that progesterone receptor antagonists may have potential applications in contraception and for the treatment of reproductive disorders such as fibroids and endometriosis. Currently, all clinically available progesterone receptor agonists and antagonists are steroidal compounds. They often cause various side effects due to their functional interactions with other steroid receptors or because of effects associated with their steroidal metabolites {Winneker, Richard C. et al.; Endocrin. and Repro. Disorders Div., Women's Health Research Institute, Collegeville, Pa., USA. Seminars in Reproductive Medicine (2005), 23(1), 46-57}.

Progesterone receptor antagonists [anti-progestins (APs)], including the founding members of the class mifepristone (RU-486; Roussel UCLAF, Romainville, France), onapristone (ZK 98 299; Schering AG), ZK 137 316 and ZK-230 211, are compounds that bind to the progesterone receptor (PR) and prevent progesterone-induced gene expression {Spitz, I. M. (2003). Steroids, 68, 981-993.}. Acting on the estrogen primed endometrium, progesterone plays an essential role in the differentiation and ductal morphogenesis of endometrial tissue, but also participates in the inhibition of myometrial contractility and the polarisation of leukocyte Th1/Th2 responses that are critical for embryo implantation and the maintenance of pregnancy. A number of studies have investigated the potential beneficial effects of anti-progestins on the signs and symptoms of endometriosis {Grow, D. R., et al. (1996). J. Clin. Endocrin. & Metabol., 81, 1933-1939.; Kettel, L. M., et al. (1996). Fertil. & Steril., 65, 23-28.; Kettel, L. M., et al. (1998). Am. J. Obstet. & Gyn., 178, 1151-1156.} and uterine fibroids {Eisinger, S. H., et al. (2003). Obstet. & Gyn., 101, 243-250.; Murphy, A. A., and Castellano, P. Z. (1994). Curr. Opinion in Obstet. & Gyn., 6, 269-278.; Murphy, A. A., et al. (1995). Fertil. & Steril., 63, 761-766.; Steinauer, J., Pritts, et al. (2004). Obstet. & Gyn., 103, 1331-1336.; Yang, Y., et al. (1996). Chinese. Chung-Hua Fu Chan Ko Tsa Chih [Chinese J. Obstet. & Gyn.] 31, 624-626}. Unlike GnRH analogues, and other conventional pharmacological approaches, anti-progestins, especially mifepristone, appear to be able to reduce lesion or fibroid volume, whilst maintaining a tonic level of ovarian oestrogen secretion. Such anti-progestins induce amenorrhoea and endometrial compaction, and also appear to sufficiently protect against rapid oestrogen-dependent bone loss {Grow, D. R., et al. (1996). J. Clin. Endocrin. & Metabol., 81, 1933-1939.}. In contrast GnRH analogues cause a rapid loss in bone mineral density, a clinical feature which limits their treatment duration to 6 months. Whilst mifepristone is a potent anti-progestin, it also has equipotent anti-glucocorticoid activity. Outside of a palliative treatment of hypercortisolism for Cushing's syndrome {Chu, J. W., et al. (2001). J. Clin. Endocrinol. Metab. 86, 3568-3573.; Sartor, O., and Cutler, G. B., Jr. (1996). Clin. Obstet. Gynaecol. 39, 506-510.; Spitz, I. M. (2003). Steroids 68, 981-993.; Van Look, P. F., and von Hertzen, H. (1995). Human Reproduction Update 1, 19-34.}, the anti-glucocorticoid activity is an undesirable feature of mifepristone and potentially many of the steroidal classes of anti-progestins.

A further class of steroidal and non-steroidal compounds, termed the progesterone receptor modulators (PRMs, or mesoprogestins), including asoprisnil (J867, benzaldehyde, 4-[(11β, 17β)-17-methoxy-17-(methoxymethyl)-3-oxoestra-4,9-dien-11-yl]-, 1-oxime; Jenpharm, TAP), J912, J956, J1042, have also been described. In addition to their potential utility in hormone replacement and as contraceptives, these classes of compounds could be considered to have utility in the treatment of endometriosis and uterine leiomyoma {Chwalisz, K., et al. (2004). Semin. Reprod. Med., 22, 113-119.; Chwalisz, K., et al. (2002). Ann. NY Acad. of Sci., 955, 373-388; discussion 389-393.; DeManno, D., et al. (2003). Steroids 68, 1019-1032.}. Asoprisnil and structurally-related PRMs differ from anti-progestins and progestins in animal models, demonstrating partial progestogenic activity in the rabbit endometrium (McPhail's test {McPhail, M. K. (1934). J. Physiol., 83, 145-156.}) and guinea pig vagina, for instance. Pre-clinical studies with asoprisinil in primates have indicated that PRMs suppress endometrial growth and, unlike the effects of progestins, endometrial ER and PR expression is not repressed {Chwalisz, K., et al. (2000). Steroids, 65, 741-751.; DeManno, D., et al. (2003). Steroids, 68, 1019-1032.; Elger, W., et al. (2000). Steroids, 65, 713-723.}.

U.S. Pat. Appl'n. Pub. No. 2006/0020012 describes pyrazole derivatives of the formula:

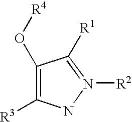

wherein R¹, R², R³ and R⁴ are as defined therein, which are modulators of HIV reverse transcriptase. U.S. Pat. Appl'n. Pub. Nos. 2006/0241125 and 2007/0105909; and U.S. Prov. Pat. Appl'n. Nos. 60/862,126, filed 19^{th} Oct. 2006, and 60/862,136 filed 19^{th} Oct. 2006, all disclose 4-(4-cyanophenyloxy)pyrazole compounds with progesterone antagonist activity.

### DETAILED DESCRIPTION OF THE INVENTION

The compounds of the present invention are useful in treating conditions such as endometriosis, uterine fibroids (leiomyomata) and menorrhagia, adenomyosis, primary and secondary dysmenorrhoea (including symptoms of dyspareunia, dyschexia and chronic pelvic pain), chronic pelvic pain syndrome, precocious puberty, cervical ripening, contraception (emergency), breast carcinoma, ovarian carcinoma, endometrial carcinoma, prostate carcinoma, pulmonary carcinoma, testicular carcinoma, gastric carcinoma, meningioma, anxiety, premenstrual syndrome, premenstrual dysphoric disorder, alcohol abuse and reward, or Charcot-Marie-Tooth disease. Particularly of interest are the following diseases or disorders: endometriosis, uterine fibroids (leiomyomata), menorrhagia, adenomyosis, primary and secondary dysmenorrhoea (including symptoms of dyspareunia, dyschexia and chronic pelvic pain), and chronic pelvic pain syndrome.

In particular, the compounds and derivatives of the present invention exhibit activity as progesterone receptor antagonists and may be useful for treatment where progesterone receptor antagonism is indicated.

More particularly, the compounds and derivatives of the present invention may be useful for treating endometriosis and/or uterine fibroids (leiomyomata).

According to the present invention there is provided a compound of Formula (I)

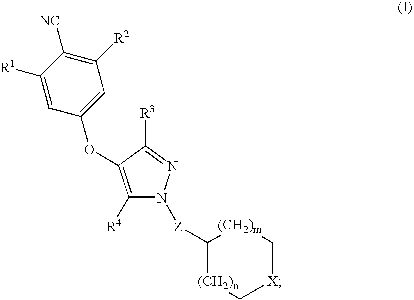

and the pharmaceutically acceptable salts, solvates (including hydrates) of said compounds and salts, and prodrugs of said compounds, and pharmaceutically acceptable salts and solvates of said prodrugs thereof, wherein:
R¹ and R² are independently selected from H, C₁₋₆alkyl optionally substituted by one or more halogen, C₁₋₆alkyloxy optionally substituted by one or more halogen, CN, and halo;
R³ is selected from the group consisting of H; C₁₋₆alkyl; C₁₋₆alkyloxy; C₃₋₈cycloalkyl; and halo;
R⁴ is C₁₋₆alkyl optionally substituted by one or more fluorine and/or nitrile groups, C₁₋₆alkyloxy, C₃₋₈cycloalkyl optionally substituted by one or more fluorine and/or nitrile groups, or halo;
X represents O, S, S(O) or SO₂;
Z represents a bond or (CR⁵R⁶); where R⁵ and R⁶ are independently selected from the group consisting of H; and C₁₋₆alkyl; or R⁵ and R⁶ together with the carbon to which they are attached form a 3 to 6 membered carbocyclic ring which optionally bears from 1 to 3 substituents independently selected from the group consisting of halo; cyano; hydroxyl; C₁₋₄alkyl; C₁₋₄haloalkyl; C₁₋₄haloalkyloxy and C₁₋₄alkyloxy; and
m and n independently can be 0, 1 or 2 provided that m+n is not more than 3;
and wherein said alkyl, cycloalkyl or alkoxy groups may be independently optionally (further) substituted with from 1 to 5 substituents independently chosen from the group consisting of halo, cyano, hydroxyl, C₁₋₄alkyl; C₁₋₄haloalkyl; C₁₋₄haloalkyloxy and C₁₋₄alkyloxy.

Preferably R¹ is H, Cl, C₁₋₃ alkyl optionally substituted by one or more halogen or C₁₋₃ alkyloxy optionally substituted by one or more halogen. More preferably R¹ is H, Cl, CH₃, CF₃, OCF₃ or OCH₃; particularly R¹ is H, Cl, CH₃. Most preferably R¹ is CH₃.

Preferably R² is H, Cl, C₁₋₃ alkyl optionally substituted by one or more halogen or C₁₋₃ alkyloxy optionally substituted by one or more halogen. More preferably R² is H, Cl, CH₃, CF₃, OCF₃ or OCH₃; particularly R² is H, Cl, CH₃. Most preferably R² is CH₃.

In a preferred group, both R¹ and R² are independently selected from H, Cl, CH₃, CF₃, OCF₃ and OCH₃.

In a more preferred group,
R¹ is H and R² is CH₃,
R¹ is H and R² is Cl,
R¹ is H and R² is CF₃,
R¹ is H and R² is OCH₃,
R¹ and R² are both CH₃ and
R¹ and R² are both H.

Particularly R¹ and R² are both CH₃, or R¹ is H and R² is either H or Cl., CH₃

Preferably R³ is selected from the group comprising: methyl; ethyl; cyclopropyl; and chloro. Most preferably, R³ is methyl or cyclopropyl.

Preferably R⁴ is C₁₋₆ alkyl or C₃₋₈ cycloalkyl. More preferably R⁴ is C₁₋₄ alkyl or C₃₋₄ cycloalkyl. Yet more preferably R⁴ is C₃₋₄ alkyl or C₃₋₄ cycloalkyl. Even yet more preferably R⁴ is methyl, isopropyl, cyclopropyl or cyclobutyl. Most preferably R⁴ is cyclopropyl.

Preferably Z is a bond or (CH₂). More preferably Z is a bond.

Preferably m+n is zero, or m is 1 and n is 1 or m is 0 and n is 1.

A preferred group of compounds, pharmaceutically acceptable salts, solvates (including hydrates), and prodrugs thereof are those wherein:
R¹ is H, Cl, CF₃, CH₃, CF₃ or OCF₃;
R² is H, Cl, CF₃, CH₃, CF₃ or OCF₃;
R³ is methyl or cyclopropyl;
R⁴ is isopropyl, cyclopropyl or cyclobutyl;
Z is a bond or CH₂;
and m+n is zero, or m is 1 and n is 1.

Preferably X is O or SO₂.

More preferably X is SO₂.

A further preferred group of compounds, pharmaceutically acceptable salts, solvates (including hydrates), and prodrugs thereof are those wherein:
R¹ is H, Cl or CH₃,
R² is H, Cl or CH₃,
R³ is methyl or cyclopropyl;
R⁴ is hydrogen, methyl, cyclopropyl or cyclobutyl;
Z is a bond;
X is O, SO or SO₂; and
m+n is zero, or m is 1 and n is 1 or m is 0 and n represents 1.

In the above definitions alkyl groups containing the requisite number of carbon atoms, except where indicated, can be unbranched or branched chain. Examples include methyl, ethyl, n-propyl, i-propyl, n-butyl, i-butyl, sec-butyl and t-butyl. Examples of alkyloxy include methoxy, ethoxy, n-propyloxy, i-propyloxy, n-butyloxy, i-butyloxy, sec-butyloxy and t-butyloxy. Examples of cycloalkyl include cyclopropyl, cyclobutyl, cyclopentyl, cyclohexyl and cycloheptyl. The term halogen means fluoro, chloro, bromo or iodo.

Pharmaceutically acceptable derivatives of the compounds of formula (I) according to the invention include salts, solvates, complexes, polymorphs and crystal habits thereof, prodrugs, stereoisomers, geometric isomers, tautomeric forms, and isotopic variations of compounds of formula (I). Preferably, pharmaceutically acceptable derivatives of compounds of formula (I) comprise salts, solvates, esters and amides of the compounds of formula (I). More preferably, pharmaceutically acceptable derivatives of compounds of formula (I) are salts and solvates.

The pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemi-salts of acids and bases may also be formed, for example, hemi-sulphate and hemicalcium salts. For a review on suitable salts, see “Handbook of Pharmaceutical Salts: Properties, Selection, and Use” by Stahl and Wermuth (Wiley-VCH, 2002).

Pharmaceutically acceptable salts of compounds of formula I may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see “Polymorphism in Pharmaceutical Solids” by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem. Comm., 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

As indicated above, so-called ‘prodrugs’ of the compounds of formula (I) are also within the scope of the invention. Thus certain derivatives of compounds of formula (I), which may have little or no pharmacological activity themselves, can be converted into compounds of formula I having the desired activity, for example by hydrolytic cleavage, when administered into, or onto, the body. Such derivatives are referred to as ‘prodrugs’. Further information on the use of prodrugs may be found in “Pro-drugs as Novel Delivery Systems”, Vol. 14, ACS Symposium Series (T. Higuchi and W. Stella) and “Bioreversible Carriers in Drug Design”, Pergamon Press, 1987 (Ed. E. B. Roche, American Pharmaceutical Association).

Prodrugs in accordance with the invention can be produced by replacing appropriate functionalities present in the compounds of formula (I) with certain moieties known to those skilled in the art as ‘pro-moieties’ as described, for example, in “Design of Prodrugs” by H. Bundgaard (Elsevier, 1985).

Some examples of prodrugs in accordance with the invention include:
(i) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(ii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references. Moreover, certain compounds of formula (I) may themselves act as prodrugs of other compounds of formula (I).

Also included within the scope of the invention are metabolites of compounds of formula (I), that is, compounds formed in vivo upon administration of the drug. Thus within the scope of the invention are envisaged the metabolites of the compounds of formula (I) when formed in vivo.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

The compounds of the invention may be administered alone or in combination with one or more other compounds of the invention or in combination with one or more other drugs (or as any combination thereof).

The compounds of the present invention may be administered in combination with COX inhibitors. Thus in a further aspect of the invention, there is provided a pharmaceutical product containing a progesterone receptor antagonist and one or more COX inhibitors as a combined preparation for simultaneous, separate or sequential use in the treatment of endometriosis. COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al., Europ. J. Pharmacol. 453:319-324 (2002));
(ii) meloxicam, (CAS registry number 71125-38-7; U.S. Pat. No. 4,233,299), or a pharmaceutically acceptable salt or prodrug thereof;
(iii) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (WO 98/03484), JTE-522 (Japanese Patent Application Publication No. 9052882), or a pharmaceutically acceptable salt or prodrug thereof;
(iv) Parecoxib (U.S. Pat. No. 5,932,598), which is a therapeutically effective prodrug of the tricyclic Cox-2 selective inhibitor valdecoxib (U.S. Pat. No. 5,633,272), in particular sodium parecoxib;
(v) ABT-963 (WO 00/24719)
(vi) Nimesulide (U.S. Pat. No. 3,840,597), flosulide (J. Carter, Exp. Opin. Ther. Patents. 8(1), 21-29 (1997)), NS-398 (U.S. Pat. No. 4,885,367), SD 8381 (U.S. Pat. No. 6,034,256), BMS-347070 (U.S. Pat. No. 6,180,651), S-2474 (EP 595,546) and MK-966 (U.S. Pat. No. 5,968,974);
(vii) darbufelone (Pfizer), CS-502 (Sankyo), LAS 34475 (Almirall Profesfarma), LAS 34555 (Almirall Profesfarma), S-33516 (Servier), SD 8381 (Pharmacia, U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

The compounds of the present invention may be administered in combination with PDE5 inhibitors. Thus in a further aspect of the invention, there is provided a pharmaceutical product containing a progesterone receptor antagonist and one or more PDEV inhibitors as a combined preparation for simultaneous, separate or sequential use in the treatment of endometriosis. PDEV inhibitors useful for combining with compounds of the present invention include, but are not limited to:
(i) Preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxy phenyl]-2-(pyridin-2-yl)methyl-2,6-dihydro-7H-pyrazolo[4,3-d]pyrimidin-7-one (see WO 98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo [4,3-d]pyrimidin-7-one(see WO99/54333); (+)-3-ethyl-5-[5-(4-ethylpiperazin-1-ylsulphonyl)-2-(2-methoxy-1(R)-methylethoxy) pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d] pyrimidin-7-one (see WO99/54333); 5-[2-ethoxy-5-(4-ethylpiperazin-1-ylsulphonyl)pyridin-3-yl]-3-ethyl-2-[2-methoxy ethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl)-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d] pyrimidin-7-one(see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl) pyrazino[2′, 1′:6,1]pyrido[3,4-b]indole-1,4-dione (tadalafil, IC-351, Cialis®), i.e. the compound of examples 78 and 95 of WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®)) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of WO99/24433; the compound of example 11 of WO93/07124 (EISAI); compounds 3 and 14 from Rotella D P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl-2-pyrrolidine propanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl) methyl]-7,8-dihydro-8-oxo-1H-imidazo[4,5-g]quinazolin-6-yl]-4-propoxyphenyl]carboxamide; 4-[(3-chloro-4-methoxybenzyl)amino]-2-[(2S)-2-(hydroxymethyl)pyrrolidin-1-yl]-N-(pyrimidin-2-yl methyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzene sulfonamide (DA 8159) and pharmaceutically acceptable salts thereof.
(ii) 4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, mono-sodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propyl indole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

Preferably the PDEV inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one. Most preferably the PDE5 inhibitor is sildenafil and pharmaceutically acceptable salts thereof. Sildenafil citrate is a preferred salt.

The compounds of the present invention may be administered in combination with a V1a antagonist. Thus, in a further aspect of the invention, there is provided a pharmaceutical product containing a progesterone receptor antagonist and one or more V1a antagonists as a combined preparation for simultaneous, separate or sequential use in the treatment of endometriosis.

A suitable vasopressin V1a receptor antagonist is, for example, (4-[4-Benzyl-5-(4-methoxy-piperidin-1-ylmethyl)-4H-[1,2,4]triazol-3-yl]-3,4,5,6-tetrahydro-2H-[1,2′]bipyridinyl), which is Example 26 in WO 2004/37809. A further example of a suitable vasopressin V1a receptor antagonist is 8-chloro-5-Methyl-1-(3,4,5,6-tetrahydro-2H-[1,2′]bipyridinyl-4-yl)-5,6-dihydro-4H-2,3,5,10b-tetraazo-benzo[e]azulene, or a pharmaceutically acceptable salt or solvate thereof, which is Example 5 in WO 04/074291.

Further examples of vasopressin V1a receptor antagonists for use with the invention are: SR49049 (Relcovaptan), atosiban (Tractocilei), conivaptan (YM-087), VPA-985, CL-385004, Vasotocin and OPC21268. Additionally, the V1a receptor antagonists described in WO 01/58880 are suitable for use in the invention.

The compounds of the present invention may be administered in combination with an alpha adrenergic receptor antagonist (also known as α-adrenoceptor blocker, α-receptor blocker or α-blocker). Thus, in a further aspect of the invention, there is provided a pharmaceutical product containing a progesterone receptor antagonist and one or more alpha adrenergic receptor antagonists as a combined preparation for simultaneous, separate or sequential use in the treatment of endometriosis.

α₁-Adrenergic receptor antagonists useful for the present invention include, but are not limited to, terazosin (U.S. Pat. No. 4,026,894), doxazosin (U.S. Pat. No. 4,188,390), prazosin (U.S. Pat. No. 3,511,836), bunazosin (U.S. Pat. No. 3,920,636), alfuzosin (U.S. Pat. No. 4,315,007), naftopidil (U.S. Pat. No. 3,997,666), tamsulosin (U.S. Pat. No. 4,703,063), silodosin (U.S. Pat. No. 5,387,603), phentolamine and phentolamine mesylate (U.S. Pat. No. 2,503,059), trazodone (U.S. Pat. No. 3,381,009), indoramin (U.S. Pat. No. 3,527,761), phenoxybenzamine (U.S. Pat. No. 2,599,000), rauwolfa alkaloids (natural product from the shrub Rauwolfia serpentine), Recordati 15/2739 (WO 93/17007), SNAP 1069 (WO 94/08040 e.g. 3, compound 9, page 77 & table 3, page 86), SNAP 5089 (WO 94/10989), RS17053 (U.S. Pat. No. 5,436,264), SL 89.0591 (EP 435749), and abanoquil (EP 100200); the compounds disclosed in WO 03/076427 in particular 5-cyclopropyl-7-methoxy-2-(2-morpholin-4-ylmethyl-7,8-dihydro[1,6]-naphthyridin-6(5H)-yl)-4(3H)-quinazolinone (example 11), and the compounds disclosed in WO 98/30560 in particular 4-amino-6,7-dimethoxy-2-(5-methanesulfonamido-1,2,3,4-tetrahydroisoquinol-2-yl )-5-(2-pyridyl)quinazoline (example 19); and pharmaceutically acceptable derivatives thereof. Preferred α-adrenergic receptor antagonists are doxazosin, 5-cyclopropyl-7-methoxy-2-(2-morpholin-4-ylmethyl-7,8-dihydro[1,6]-naphthyridin-6(5H)-yl)-4(3H)-quinazolinone and 4-Amino-6,7-dimethoxy-2-(5-methanesulfonamido-1,2,3,4-tetrahydroisoquinol-2-yl)-5-(2-pyridyl)quinazoline and pharmaceutically acceptable derivatives thereof. The mesylate salt of 4-amino-6,7-dimethoxy-2-(5-methanesulfonamido-1,2,3,4-tetrahydroisoquinol-2-yl)-5-(2-pyridyl)quinazoline is of particular interest (see WO 01/64672).

α₂-Adrenergic receptor antagonists suitable for the present invention include dibenamine (DE 824208), tolazoline (U.S. Pat. No. 2,161,938), trimazosin (U.S. Pat. No. 3,669,968), efaroxan (EP 71368), yohimbine (M R Goldberg et al, Pharmacol. Rev. 35, 143-180 (1987)), idazoxan (EP 33655), and clonidine (U.S. Pat. No. 3,202,660); Non-selective α-adrenergic receptor antagonists suitable for the present invention include dapiprazole (U.S. Pat. No. 4,252,721).

The compounds of the present invention may be administered in combination with an 5-alpha reductase inhibitor. Thus, in a further aspect of the invention, there is provided a pharmaceutical product containing a progesterone receptor antagonist and one or more 5-alpha reductase inhibitors as a combined preparation for simultaneous, separate or sequential use in the treatment of endometriosis.

5-alpha reductase inhibitors include inhibitors of 5-alpha reductase isoenzyme 2. Suitable compounds for use in the present invention are PROSCAR® (also known as finasteride, U.S. Pat. Nos. 4,377,584 and 4,760,071), compounds described in WO 93/23420, EP0572166, WO 93/23050, WO 93/23038, WO 93/23048, WO 93/23041, WO 93/23040, WO 93/23039, WO 93/23376, WO 93/23419, EP0572165, and WO 93/23051.

The compounds of the present invention may be administered in combination with an agent which lowers estrogen levels, or which antagonises the estrogen receptor. Thus, in a further aspect of the invention, there is provided a pharmaceutical product containing a progesterone receptor antagonist and one or more agents which lower estrogen levels, or antagonise the estrogen receptor, as a combined preparation for simultaneous, separate or sequential use in the treatment of endometriosis.

Agents which lower estrogen levels include gonadotropin releasing hormone (GnRH) agonists, GnRH antagonists and estrogen synthesis inhibitors. Agents which antagonise the estrogen receptor, i.e. estrogen receptor antagonists, include anti-estrogens.

GnRH agonists suitable for the present invention include leuprorelin (Prostap—Wyeth), buserelin (Suprefact—Shire), goserelin (Zoladex—Astra Zeneca), triptorelin (De-capeptyl—Ipsen), nafarelin (Synarel—Searle), deslorelin (Somagard—Shire), and histrelin/supprelin (Ortho Pharmaceutical Corp/Shire). GnRH antagonists suitable for the present invention include teverelix (also known as antarelix), abarelix (Plenaxis—Praecis Pharmaceuticals Inc.), cetrorelix (Cetrotide—ASTA Medica), and ganirelix (Orgalutran—Organon).

Anti-estrogens suitable for the present invention include tamoxifen, Faslodex (Astra Zeneca), idoxifene (see Coombes et al. (1995) Cancer Res., 55, 1070-1074), raloxifene or EM-652 (Labrie, F. et al, (2001) J. Steroid Biochem. Mol. Biol., 79, 213).

Estrogen synthesis inhibitors suitable for the present invention include aromatase inhibitors. Examples of aromatase inhibitors include Formestane (4-OH androstenedione), Exemestane, Anastrozole (Arimidex) and Letroxole.

The compounds of the present invention may be administered in combination with an alpha-2-delta ligand. Thus, in a further aspect of the invention, there is provided a pharmaceutical product containing a progesterone receptor antagonist and one ore more alpha-2-delta ligands, as a combined preparation for simultaneous, separate or sequential use in the treatment of endometriosis.

Examples of alpha-2-delta ligands for use in the present invention are those compounds, or pharmaceutically acceptable salts thereof, generally or specifically disclosed in U.S. Pat. No. 4,024,175, particularly gabapentin, EP641330, particularly pregabalin, U.S. Pat. No. 5,563,175, WO-A-97/33858, WO-A-97/33859, WO-A-99/31057, WO-A-99/31074, WO-A-97/29101, WO-A-02/085839, particularly [(1R,5R,6S)-6-(aminomethyl)bicyclo[3.2.0]hept-6-yl]acetic acid, WO-A-99/31075, particularly 3-(1-aminomethyl-cyclohexylmethyl)-4H-[1,2,4]oxadiazol-5-one and C-[1-(1H-tetrazol-5-ylmethyl)-cycloheptyl]-methylamine, WO-A-99/21824, particularly (3S,4S)-(1-aminomethyl-3,4-dimethyl-cyclopentyl)-acetic acid, WO-A-01/90052, WO-A-01/28978, particularly (1α,3α,5α)(3-amino-methyl-bicyclo[3.2.0]hept-3-yl)-acetic acid , EP0641330, WO-A-98/17627, WO-A-00/76958, particularly (3S,5R)-3-aminomethyl-5-methyl-octanoic acid, WO-A-03/082807, particularly (3S,5R)-3-amino-5-methyl-heptanoic acid, (3S,5R)-3-amino-5-methyl-nonanoic acid and (3S,5R)-3-amino-5-methyl-octanoic acid, WO-A-2004/039367, particularly (2S,4S)-4-(3-fluoro-phenoxymethyl)-pyrrolidine-2-carboxylic acid, (2S,4S)-4-(2,3-difluoro-benzyl)-pyrrolidine-2-carboxylic acid, (2S,4S)-4-(3-chlorophenoxy)proline and (2S,4S)-4-(3-fluorobenzyl)proline, EP1178034, EP1201240, WO-A-99/31074, WO-A-03/000642, WO-A-02/22568, WO-A-02/30871, WO-A-02/30881 WO-A-02/100392, WO-A-02/100347, WO-A-02/42414, WO-A-02/32736 and WO-A-02/28881, all of which are incorporated herein by reference.

Preferred alpha-2-delta ligands for use in the combination of the present invention include: gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid , (3S,5R)-3-amino-5-methyl-octanoic acid, (2S,4S)-4-(3-chlorophenoxy)proline and (2S,4S)-4-(3-fluorobenzyl)proline or pharmaceutically acceptable salts thereof.

Further preferred alpha-2-delta ligands for use in the combination of the present invention are (3S,5R)-3-amino-5-methyloctanoic acid, (3S,5R)-3-amino-5-methylnonanoic acid, (3R,4R,5R)-3-amino-4,5-dimethylheptanoic acid and (3R,4R,5R)-3-amino-4,5-dimethyloctanoic acid, and the pharmaceutically acceptable salts thereof.

Particularly preferred alpha-2-delta ligands for use in the combination of the present invention are selected from gabapentin, pregabalin, (3S,5R)-3-amino-5-methyloctanoic acid, (1α,3α,5α)(3-amino-methyl-bicyclo[3.2.0]hept-3-yl)-acetic acid, (2S,4S)-4-(3-chlorophenoxy)proline and (2S,4S)-4-(3-fluorobenzyl)proline or pharmaceutically acceptable salts thereof.

The compounds of the present invention may be administered in combination with an oxytocin receptor antagonist. Thus, in a further aspect of the invention, there is provided a pharmaceutical product containing a progesterone receptor antagonist and one ore more oxytocin antagonists, as a combined preparation for simultaneous, separate or sequential use in the treatment of endometriosis. Examples of oxytocin receptor antagonists suitable for the present invention are atosiban (Ferring AB), barusiban (Ferring AB), TT-235 (Northwestern University), and AS-602305 (Serono SA).

The contents of the published patent applications mentioned above, and in particular the general formulae of the therapeutically active compounds of the claims and exemplified compounds therein, are incorporated herein in their entirety by reference thereto.

The compounds of the present invention may also be administered in combination with any one or more of the following:
(i) Aromatase inhibitor;
(ii) Estrogen receptor agonist;
(iii) Angiogenesis inhibitor;
(iv) VEGF inhibitor;
(v) Kinase inhibitor;
(vi) Protein farnesyl transferase inhibitor;
(vii) Androgen receptor modulator;
(viii) Androgen receptor agonists;
(ix) Androgen receptor antagonists;
(x) Prostanoid receptor agonist;
(xi) Prostanoid receptor antagonist;
(xi) Prostaglandin synthetase inhibitor;
(xii) Bioflavanoid;
(xiii) Alkylating agent;
(xiv) Microtobule modulator, e.g. Microtobule stabilizer;
(xv) Topoisomerase I inhibitor;
(xvi) Metalloprotease inhibitor; or
(xvii) Progesterone modulator.

Thus, in a further aspect of the invention, there is provided a pharmaceutical product containing a progesterone receptor antagonist and any one or more of the following:
(i) Aromatase inhibitor;
(ii) Estrogen receptor agonist;
(iii) Angiogenesis inhibitor;
(iv) VEGF inhibitor;
(v) Kinase inhibitor;
(vi) Protein farnesyl transferase inhibitor;
(vii) Androgen receptor modulator;
(viii) Androgen receptor agonists;
(ix) Androgen receptor antagonists;
(x) Prostanoid receptor agonist;
(xi) Prostanoid receptor antagonist;
(xi) Prostaglandin synthetase inhibitor;
(xii) Bioflavanoid;
(xiii) Alkylating agent;
(xiv) Microtobule modulator, e.g. Microtobule stabilizer;
(xv) Topoisomerase I inhibitor;
(xvi) Metalloprotease inhibitor; or
(xvii) Progesterone modulator,
   as a combined preparation for simultaneous, separate or sequential use in the treatment of endometriosis.

Generally, compounds of the invention will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents. Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Consumable oral films are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin. The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid. Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose. A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol. Suitable flavours, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, for example, PGLA. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus, the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range <1 mg to 1000 mg depending, of course, on the mode of administration. For example, oral administration may require a total daily dose of from <1 mg to 1000 mg, while an intravenous dose may only require from <1 mg to 500 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As used herein, the terms “treating” and “to treat”, mean to alleviate symptoms, eliminate the causation either on a temporary or permanent basis, or to prevent or slow the appearance of symptoms. The term “treatment” includes alleviation, elimination of causation (either on a temporary or permanent basis) of, or prevention of symptoms and disorders associated with endometriosis and/or uterine leiomyoma. The treatment may be a pre-treatment as well as a treatment at the on-set of symptoms.

The compounds of the present invention may be tested in the screens set out below:

1.0 In Vitro Functional Assay for Progesterone Receptor (PR) Antagonists, Agonists and Modulators

The assay for PR antagonism takes advantage of the extensively reported modulation of alkaline phosphatase (AP) expression in human breast T47D mammary carcinoma cells {Beck, et al., D. P. (1993). The progesterone antagonist RU486 acquires agonist activity upon stimulation of cAMP signalling pathways. Proc Natl Acad Sci U S A 90, 4441-4445; Fensome, et al. (2002). New progesterone receptor antagonists: 3,3-disubstituted-5-aryloxindoles. Bioorg. Med. Chem. Lett., 12, 3487-3490; Zhang et al., (2002a). 6-Aryl-1,4-dihydro-benzo d 1,3 oxazin-2-ones: a novel class of potent, selective, and orally active nonsteroidal progesterone receptor antagonists. J. Med. Chem., 45, 4379-4382; Zhang et al., (2003). Novel 6-aryl-1,4-dihydrobenzo d oxazine-2-thiones as potent, selective, and orally active nonsteroidal progesterone receptor agonists. Bioorg. Med. Chem. Lett., 13, 1313-1316; Zhang et al., (2002b). Potent nonsteroidal progesterone receptor agonists: synthesis and SAR study of 6-aryl benzoxazines. Bioorg. Med. Chem. Lett., 12, 787-790; Zhang, Z. et al., (2000). In vitro characterization of trimegestone: a new potent and selective progestin. Steroids 65, 637-643.}. In the presence of progesterone or progesterone receptor agonists endogenous AP expression is induced in T47D cells and is inhibited by compounds possessing PR antagonistic activity. In the absence of progesterone any agonist activity is also observed as an induction of AP activity. By running the assay in two formats (± progesterone (P4)), compounds behaving as PR antagonists, agonists, partial agonists or modulators with mixed agonist/antagonist activity can be identified.

The equipment required to grow T47D cells and perform the progesterone-induced AP assay are outlined below.

### Equipment List

Plates:
96-well v-bottom polypropylene plates Greiner 651201
384 well polypropylene plates Matrix 4314
384-well white, polypropylene lidded plates (tissue culture treated)
   Greiner 781080-PFI

PlateMate™ Plus:
0.5 μL to 30 μL DART Tips Matrix 5316

LJL Analyst:

Multidrop: with sterile head Thermolabsystems
Cedex: AS20 cell counter Innovatis

### General Lab Equipment:

Pipettes ranging from 2 μL to 5000 μL
50 mL and 15 mL centrifuge tubes
Class II laminar flow hood
−80° C. freezer

The materials required to grow T47D cells and perform the progesterone-induced AP assay are outlined in Table 1.

TABLE 1

| | | Catalogue |
|---|---|---|
| Reagent | Supplier | number |
| T47D human mammary carcinoma cells | American tissue culture collections; | HTB-133 |
| | http://www.atcc.org/ | |
| Dimethyl sulphoxide (DMSO) | Sigma | D2650 |
| Dulbecco's modified Eagle's Medium | Gibco | 21969-035 |
| (DMEM) | | |
| DMEM without phenol red | Gibco | 31053-028 |
| L-Glutamine, 200 mM | Gibco | |
| Charcoal stripped foetal calf serum (CS- | Globepharm | HYC-001- |
| FCS) | | 325B (lot. |
| | | APD21146) |
| Phosphate buffered saline (PBS) | Gibco | 14190-094 |
| Foetal bovine serum (FBS) | PAA | A15-245 |
| TROPIX CSPD Ready-to-use Emerald II | Applied Biosystems | CD100RY |
| reagent | | |
| Progesterone (P4) | Sigma | P-6149 |
| Pluronic-F127 | Molecular Probes | P6867 |
| RU486 (Mifepristone) | Sigma | M-8046 |

Growth medium: DMEM+Red (21969-035)

50 mL FCS (10%)

6 mL Glutamine (2 mM)

Assay medium: DMEM—Red (31053-028)

25 mL Charcoal Stripped FCS (5%)

6 mL Glutamine (2 mM)

Briefly, T47D cells are grown by propagating in DMEM with phenol red+10% FCS+2 mM Glutamine at 37° C./5% CO₂. At 70-80% confluence, the media is exchanged for phenol red free DMEM+5% CS-FCS (Assay media). Cells are incubated overnight in assay media then harvested and frozen in assay media containing 10% DMSO at 1.5e7 cells/mL in 2 mL aliquots using a Planer and immediately stored in liquid nitrogen. V1als are removed from liquid nitrogen storage and immediately thawed in a water bath at 37° C. The cell suspension is added dropwise to 20 mL of assay media, then the tube centrifuged at 1000 rpm for 4 minutes, the supernatant is discarded and the pellet re-suspended in 20 mL assay media. T47D cells are then plated at 8750 cells/well in 35 μL assay media in sufficient white solid 384 well TC plates for the assay. For the agonist format assay a further 10 μL of assay media is added to each well. These plates are then cultured for 3-6 hours at 37° C./5% CO₂ before compound addition.

### Preparation of Compounds

Compounds are prepared by in 100% DMSO, half log concentrations from 4 mM in 384 well plates 5 μL/well (referred to here as ‘grandmother plates’). Progesterone is 100 μM made up in ethanol and PBS (i.e. add 1 mL ethanol to progesterone initially to help dissolving) and stored in 0.5-1 mL aliquots at −20° C.

### Buffer Diluent

### PBS+0.05% Pluronic F1267 PBS+2.5% DMSO+0.05% Pluronic F1267

Using a multidrop (Thermolabsystems), 60 μL/well of buffer is added to a 384 well plate—this will be the ‘mother plate’. Add 45 μLwell buffer to grandmother plate using the multidrop (=400 μM 10% DMSO dilution). Mixed and spun to remove air bubbles.

20 μL is taken from the grandmother plate (400 μM 10% DMSO) and added to the mother plate (=100 μM 2.5% DMSO). Mixed and spun to remove air bubbles

### Preparation of Max/Min:

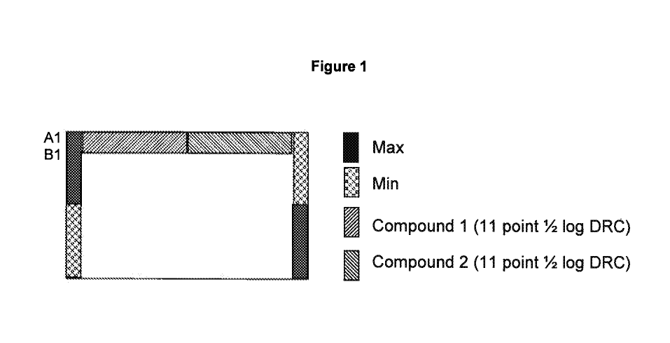

FIG. 1 is a Plate Map for use in determining Max/Min.

The Max's & Min's are prepared as below in Falcon tubes and then 100 μL is transferred to the appropriate wells of a 384 well plate:

### Agonist Max: (Solid Block on FIG. 1) 10 μM Proaesterone (FAC 1 μM)

500 μL of 100 μM progesterone

4.5 mL diluent

### Aqonist Min: (Checker Pattern on FIG. 1) Diluent

Diluent

### Antagonist Max: (Solid Block on FIG. 1) Diluent

Diluent

### Antagonist Min: (Checker Pattern on FIG. 1) 1 μM RU-486 (FAC 0.1 μM)

50 μL of 0.2 mM RU-486

10 mL Diluent

### Addition of Compound to Cell Plates

The PlateMate Plus was used to add 5 μL MAX/MIN to cell plates

Then 5 μL compounds are added from the mother plate(after removing max/min Platemate tips: columns 1 and 24)

### Addition of Agonist (5 nM Proaesterone FAC) for Antagonist Format Only

25 nM progesterone in assay media is prepared from 100 μM stock (12.5 μL progesterone/50 mL media), from which 10 μL per well is transferred to the assay plate using the Platemate Plus (already containing cells & compound).

The cell plates are incubated @ 37° C., 5% CO₂ overnight (at least 16 hours). Then:
Tap out media from cell plates and drain on tissue
Wash with PBS 40 μL/well
Tap out PBS, drain on tissue
Freeze for 15 minutes in a −80° C. freezer
Thaw in a tissue culture incubator (15 minutes)
Freeze 15 minutes in a −80° C. freezer (may be stored for at least one week without degradation of signal)
Thaw in tissue culture incubator for 5 minutes and wipe moisture from plates
Add 10 μL/well TROPIX CSPD Ready-to-use Emerald II reagent using the multidrop
Incubate 1 hour in foil at room temperature
Read on LJL Analyst luminescence counter.

In the agonist format, sigmoid fitting of the results expressed as alkaline phosphatase induction (% of maximal progesterone response) by the test compounds is achieved and an EC₅₀ is determined. In the antagonist format, results are expressed as alkaline phosphatase inhibition by the test compounds and an IC₅₀ is determined

### % Inhibition

The mean minimum is calculated and subtracted from all other readings.
The mean maximum is calculated and % inhibition calculated i.e. reading/X×100=% response (R) and 100−R=% inhibition.

The EC₅₀ value is defined as the drug concentration required to produce a 50% induction of AP activity compared with 5 nM progesterone alone. Compounds with full agonism achieve 100% of the response of progesterone whereas partial agonists induce AP activity to a level which is sub-maximal to that induced by progesterone. In the antagonist format, the IC₅₀ value is defined as the drug concentration required to produce a 50% inhibition of AP activity compared with 5 nM progesterone alone. For the purposes of compounds exemplified here, the IC₅₀ values are less than 5 μM. In a preferred embodiment, the IC₅₀ value is less than 500 nM. In a more preferred embodiment, the IC₅₀ is less than 50 nM.

The compounds of the invention may have the advantage that they are more potent, have a longer duration of action, have a broader range of activity, are more stable, have fewer side effects or are more selective, or have other more useful properties than the compounds of the prior art.

Thus, the invention provides:
(i) a compound of formula (I) or a pharmaceutically acceptable derivative thereof;
(ii) a process for the preparation of a compound of formula (I) or a pharmaceutically acceptable derivative thereof;
(iii) a pharmaceutical formulation including a compound of formula (I) or a pharmaceutically acceptable derivative thereof, together with a pharmaceutically acceptable excipients, diluent or carrier;
(iv) a compound of formula (I) or a pharmaceutically acceptable derivative or composition thereof, for use as a medicament;
(v) the use of a compound of formula (I) or of a pharmaceutically acceptable derivative or composition thereof, for the manufacture of a medicament for the treatment of endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome;
(vi) use as in (v) where the disease or disorder is endometriosis and/or uterine fibroids (leiomyomata);
(vii) a method of treatment of a mammal to treat endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome including treating said mammal with an effective amount of a compound of formula (I) or with a pharmaceutically acceptable derivative or composition thereof;
(viii) a method as in (vii) where the disease or disorder is endometriosis and/or uterine fibroids (leiomyomata);
(ix) novel intermediates disclosed herein; and
(x) other aspects of the invention apparent from the claims.

The routes below, including those mentioned in the Examples and Preparations, illustrate methods of synthesising compounds of formula (I) and certain derivatives thereof. The skilled person will appreciate that the compound (I) or derivative as herein defined of the invention, and intermediates thereto, could be made by methods other than those specifically described herein, for example by adaptation of the methods described herein, for example by methods known in the art. Suitable guides to synthesis, functional group interconversions, use of protecting groups, etc., are for example:
“Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

In the following general methods the substituents are as previously defined for a compound of formula (I) or derivative as herein defined unless otherwise stated.

In Scheme 1 below, compounds of formula (I) may be prepared by alkylation of a compound of formula (II) with a compound of formula (Ill) where Y is a suitable leaving group in the presence of a base. The Y group in the compound of formula (Ill) is preferably a halide or sulfonate ester such as a para-toluenesulfonate (OTs). Such compounds may be prepared according to the literature (for X═SO2 Y═Br Synthesis 1982, 7, 582, for X═SO₂ Y═OTs J. Het. Chem. 1978, 15, 515, for X═O Y═Br or I J. Org. Chem. 1973, 38, 2061 for X═O Y═OTs J. Org. Chem. 1983, 48, 2953). In a typical procedure, a solution of the compound of formula (II) in a suitable solvent is treated with a strong base and then a compound of formula (III) at a temperature between room temperature and reflux temperature of the solvent. In a preferred procedure a compound of formula (II) is treated with sodium hydride in tetrahydrofuran and then a compound of formula (III) at reflux.

Compounds of formula (II) may be prepared by condensation of a compound of formula (IV) with hydrazine or a salt or hydrate thereof, optionally in the presence of an acid or a base. The base is preferably a tertiary amine base, such as triethylamine. The acid is preferably acetic acid. In a typical procedure, a solution of the compound of formula (IV) in a suitable solvent, such as ethanol, is treated with hydrazine, or the salt or hydrate thereof, and, if used, the appropriate acid or base, at a temperature of from room temperature to the reflux temperature of the solvent. In a preferred procedure, the reaction mixture is heated under reflux.

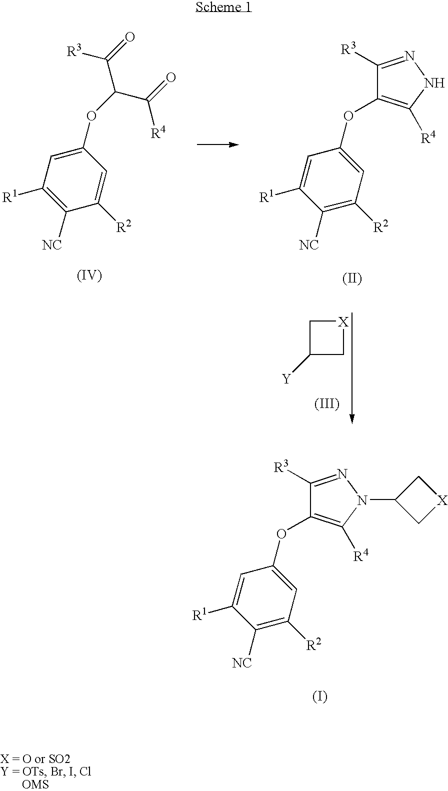

Alternatively in Scheme 2 below compounds of formula (I) where Z is a bond may be prepared by condensation of a diketone of formula (IV) with a substituted hydrazine of formula (V) in the presence of an acid or base in a suitable solvent. In a typical procedure a solution of the compound of formula (IV) in a suitable solvent is treated with a substituted hydrazine of formula (V) in the presence of an acid at elevated temperature. In a preferred procedure a solution of the compound of formula (IV) in methanol is treated with a hydrazine of formula (V) in the presence of hydrochloric acid at 60° C. In the case of substituted hydrazines of formula (V) where X═S then compounds of formula (I) where X═SO or X═SO₂ may be prepared by a treatment of compounds of formula (I) where X═S with a suitable oxidising reagent in a suitable solvent. In a typical procedure a compound of formula (I), X═S is treated with oxone in methanol at room temperature. Hydrazines of formula (V) are either commercially available, in the case of X═SO₂, n=1, m=0, or are known in the literature (e.g. X═O, n=1, m=0 U.S. Pat. No. 5,294,612; X═O, n=m=1 and X═S n=m=1 J. Med. Chem. 2004, 47, 2180-2193, X═S, n=m=1.) or available by suitable adaptation of known methods.

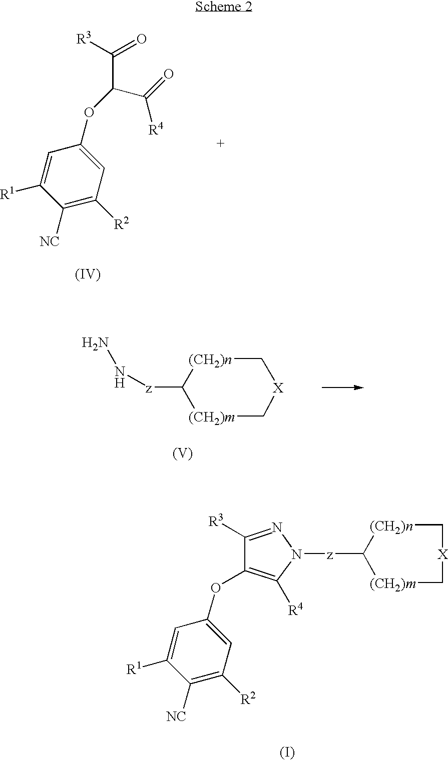

Functional equivalents of compounds of formula (IV) may also be used in these reactions. These include compounds of formula (VI) or (VII) below, in which L¹ and L², respectively, are each suitable leaving groups; preferably —N(C₁-C₆ alkyl)₂, more preferably —N(CH₃)₂.

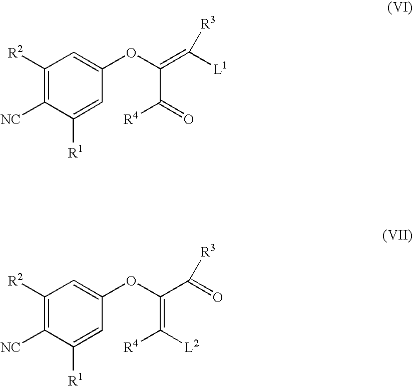

Thus, a compound of formula (I) may be prepared by the condensation of a compound of formula (VI), or (VIl), with hydrazine (V) or a salt or hydrate thereof, optionally in the presence of an acid or a base (the base preferably being a tertiary amine base, such as triethylamine, and the acid preferably being acetic acid). In a typical procedure, a solution of the compound of formula (VI), or (VII), in a suitable solvent (such as ethanol) is treated with hydrazine (V), or a salt or hydrate thereof, and, if used, the appropriate acid or base, at a temperature of from room temperature to the reflux temperature of the solvent. In a preferred procedure, the reaction mixture is heated under reflux. Compounds of formula (VI), or (VII), are particularly suitable for the synthesis of compounds of formula (I), in which R³, or R⁴, respectively, represents H by the condensation of a compound of formula (VI) or (VII) with a substituted hydrazine of formula (V).

Furthermore in Scheme 3 below compounds of formula (I) wherein one or both of R³ or R⁴ represents halogen may be prepared from compounds of formula (I) wherein the corresponding R³ and/or R⁴ represents hydrogen by a halogenation reaction. In a typical procedure, a solution of the compound of formula (I) in which one of R³ or R⁴ represents hydrogen in a suitable solvent is treated with a halogenation agent such as an N-halo succinimide at a temperature of from room temperature to the reflux temperature of the solvent. In a preferred procedure to afford compounds of formula (I) where R³ and/or R⁴ represents chloro, then a solution of the compound of formula (I) where R³ and/or R⁴ represents hydrogen in acetonitrile is treated with N-chlorosuccinimide (NCS) at 60° C.

Compounds of formula (VI) in which R³ is H and L¹ is dimethylamino may be prepared by the reaction of a compound of formula (X) or (XI), below, with dimethylformamide dimethylacetal at an elevated temperature, preferably at about 100° C. Other compounds of formula (VI) in which L¹ or L² is dimethylamino, may be prepared analogously.

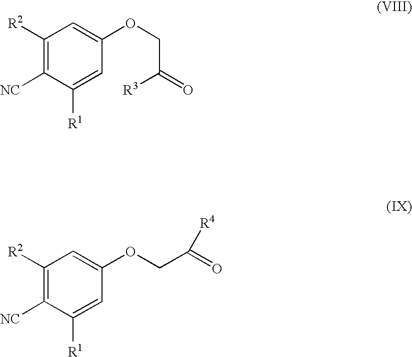

Compounds of formula (VIII) are either commercially available or may be prepared by the reaction of a compound of formula (X):

R³COCH₂Br (X)

with a compound of formula (XI):

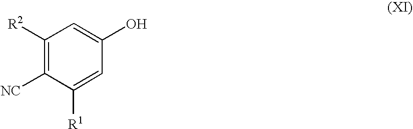

In a typical procedure, a solution of the compound of formula (XI), in a suitable solvent, such as acetone, is treated with a suitable base, such as caesium carbonate, and the compound of formula (X). In a preferred procedure, the reaction mixture is heated, for example under reflux. Optionally, a nucleophilic catalyst, such as sodium iodide or tetrabutylammonium iodide, may be added.

Compounds of formula (IX) are either commercially available or may be prepared from a compound of formula (XII):

R⁴COCH₂Br (XII)

and a compound of formula (XI), in the same way that a compound of formula (VIII) may be prepared from a compound of formula (X).

According to Scheme 4 compounds of formula (IV) may be prepared by reaction of a compound of formula (XIII) with a compound of formula (XI). In a typical procedure, a solution of the compound of formula (XIII), in a suitable solvent, such as acetone, is treated with a compound of formula (XI) and a suitable base, such as potassium or caesium carbonate, and heated, preferably under reflux. Optionally, a nucleophilic catalyst such as sodium iodide, or tetrabutylammonium iodide, may be added.

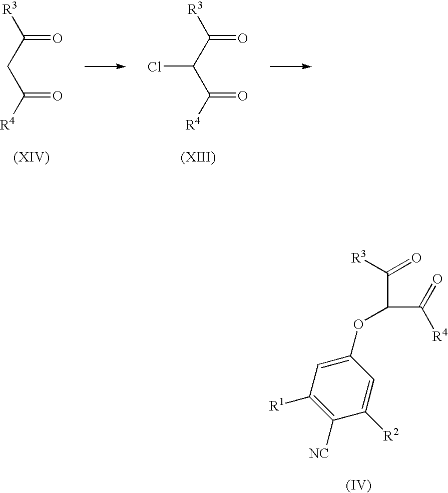

Compounds of formula (XIII) are either commercially available or may be prepared by the reaction of a compound of formula (XIV) with a chlorinating reagent. In a typical procedure, a cooled solution of the compound of formula (XIV), in a suitable solvent, such as acetonitrile, is treated first with tetrabutylammonium bromide and chlorotrimethylsilane, and then dry dimethylsulphoxide. In another typical procedure, the compound of formula (XIV) is treated with sulphuryl chloride, optionally in the presence of a suitable solvent, such as dichloromethane. In another typical procedure the compound of formula (XIV) is treated with chlorotrimethylsilane and N-chlorosuccinimide.

The following Preparations and Examples illustrate the preparation of the compounds of formula (I). The Preparations and Examples that follow illustrate the invention but do not limit the invention in any way. All starting materials are available commercially or described in the literature. All temperatures are in 0° C. Flash column chromatography was carried out using Merck silica gel 60 (9385). Thin layer chromatography (TLC) was carried out on Merck silica gel 60 plates (5729). “R_{f}” represents the distance travelled by a compound divided by the distance travelled by the solvent front on a TLC plate. Melting points were determined using a Gallenkamp MPD350 apparatus and are uncorrected. NMR was carried out using a Varian-Unity Inova 400 MHz NMR spectrometer or a Varian Mercury 400 MHz NMR spectrometer. Where high performance liquid chromatography mass spectroscopy (LCMS) has been used it refers to a Waters ZQ ESCI LC-MS system using a C18 phase Phenomenex Gemini 50×4.6 mm with 5 micron particle size column and using a gradient of 95-5% water in acetonitrile (with 0.1% formic acid) run over 3 minutes followed by a 1 minute hold and a flow rate of 1 mL/min. Where low resolution mass spectroscopy (LRMS) has been used it refers to a Waters ZQ ESCI MS system.

¹H-nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad.

The following abbreviations have been used throughout:
HRMS high resolution mass spectrometry;
LRMS low resolution mass spectrometry;
hplc high performance liquid chromatography;
nOe nuclear Overhauser effect;
m.p melting point;
CDCl₃ deuterochloroform;
D₆-DMSO deuterodimethylsulphoxide;
CD₃OD deuteromethanol

Certain compounds of the Examples and Preparations were purified using Automated Preparative High Performance Liquid Chromatography (HPLC). Reversed-phase HPLC conditions were on FractionLynx systems. Samples were submitted dissolved in 1 mL of DMSO. Depending on the nature of the compounds and the results of a pre-analysis, the purification was performed under either acidic conditions or basic conditions at ambient temperature. Acidic runs were carried out on a Sunfire Prep C18 OBD column (19'50 mm, 5 μm), basic runs were carried out on a Xterra Prep MS C18 (19×50 mm, 5 μm), both from Waters. A flow rate of 18 mL/min was used with mobile phase A: water+0.1% modifier (v/v) and B: acetonitrile+0.1% modifier (v/v). For acidic runs the modifier was formic acid, for basic run the modifier was diethylamine. A Waters 2525 binary LC pump supplied a mobile phase with a composition of 5% B for 1 min then ran from 5% to 98% B over 6 min followed by a 2 min hold at 98% B. Detection was achieved using a Waters 2487 dual wavelength absorbance detector set at 225 nm followed in series by a Polymer Labs PL-ELS 2100 detector and a Waters ZQ 2000 4 way MUX mass spectrometer in parallel. The PL 2100 ELSD was set at 30° C with 1.6 L/min supply of Nitrogen. The Waters ZQ MS was tuned with the following parameters:

ES+Cone voltage: 30 v Capillary: 3.20 kv

ES−Cone voltage: −30 v Capillary: −3.00 kv

Desolvation gas: 600 L/hr

Source Temp: 120° C.

Scan range 150-900 Da

The fraction collection was triggered by both MS and ELSD.

Quality control analysis was performed using a LCMS method orthogonal to the preparative method. Acidic runs were carried out on a Sunfire C18 (4.6×50 mm, 5 μm), basic runs were carried out on a Xterra C18 (4.6×50 mm, 5 μm), both from Waters. A flow rate of 1.5 mL/min was used with mobile phase A: water+0.1% modifier (v/v) and B: acetonitrile+0.1% modifier (v/v). For acidic runs the modifier was formic acid, for basic run the modifier was diethylamine. A Waters 1525 binary LC pump ran a gradient elution from 5% to 95% B over 3 min followed by a 1 min hold at 95% B. Detection was achieved using a Waters MUX UV 2488 detector set at 225 nm followed in series by a Polymer Labs PL-ELS 2100 detector and a Waters ZQ 2000 4 way MUX mass spectrometer in parallel. The PL 2100 ELSD was set at 30° C. with 1.6 L/min supply of Nitrogen. The Waters ZQ MS was tuned with the following parameters:

ES+Cone voltage: 25 v Capillary: 3.30 kv

ES−Cone voltage: −30 v Capillary: −2.50 kv

Desolvation gas: 800 L/hr

Source Temp: 150° C.

Scan range 160-900 Da

Where it is stated that compounds were prepared in the manner described for an earlier Preparation or Example, the skilled person will appreciate that reaction times, number of equivalents of reagents and reaction temperatures may be modified for each specific reaction, and that it may nevertheless be necessary or desirable to employ different work-up or purification conditions.

### Preparation 1: 1,3-Dicyclopropyl-propane-1,3-dione

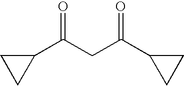

Methylcyclopropanecarboxylate (20.2 mL, 286.3 mmol) was added to a stirred solution of 1-cyclopropylethanone (9 mL, 152.4 mmol) in dimethylsulfoxide (25 mL). Sodium methoxide powder (10.8 g, 200 mmol) was added, and the reaction was stirred at 55° C. for 8 hours. The reaction mixture was then cooled, diluted with toluene (200 mL), neutralised with 6M hydrochloric acid (50 mL), separated and then extracted with toluene (100 mL). The combined extracts were washed with sodium carbonate (150 mL), dried over magnesium sulphate and evaporated in vacuo to provide the title compound (14.9 g, 78%) as a mixture 2:1 enol:ketone forms. ¹H NMR (CDCl₃, 400 MHz) δ=0.79-0.87(m, 4H), 0.98-1.01(m, 4H), 1.46-1.51 (m, 2H-enol), 1.93-1.97(m, 2H-keto), 3.70(s, 2H-keto), 5.65(s, 1H-enol); LRMS APCI⁺ m/z 153 [MH⁺]; APCI⁻ m/z 151 [M-H]⁻

### Preparation 2a: 2-Chloro-1,3-dicyclopropyl-1,3-propanedione

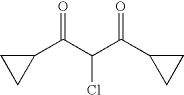

Chlorotrimethylsilane (36 mL, 296 mmol) was added dropwise to a stirred solution of tetrabutylammonium bromide (1.54 g, 5 mmol) in dry acetonitrile (100 mL) at room temperature, under nitrogen. The resulting solution was cooled in ice, and the diketone described in Preparation 1 (15 g, 98.7 mmol) as a solution in acetonitrile (30 mL) was added dropwise, followed by dry dimethylsulphoxide (20 mL, 296 mmol). The reaction mixture was allowed to warm slowly to room temperature, and then stirred for 18 hours. The reaction mixture was diluted with water (200 mL), stirred for 10 minutes and then extracted with diethyl ether (50 mL). The layers were separated, and the aqueous layer was extracted again with diethyl ether (100 mL). The organic layers were combined, dried over magnesium sulphate, filtered and concentrated under reduced pressure. The crude product was purified by column chromatography on silica gel eluting with pentane:diethyl ether (20:1, by volume) to provide the title compound as a 2:7 mixture of keto:enol tautomers (12.1 g, 66%). ¹H NMR (400 MHz, CDCl₃) δ=1.01-1.07(m, 4H), 1.16-1.21(m, 4H), 2.23-2.28(m, 2H-keto), 2.39-2.44(m, 2H-enol), 5.07(s, 1H-keto); LRMS APCI⁺ m/z 187 [MH⁺]; APCI⁻ m/z 185 [M-H]⁻

### Preparation 2b: 2-Chloro-1,3-dicyclopropyl-1,3-propanedione

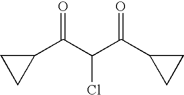

The diketone described in Preparation 1 (700 g, 4.6 mol) was dissolved in dichloromethane (7 L) with chlorotrimethylsilane (549 g, 5.08 mol), at room temperature. The solution was stirred for 45 minutes, after which time it was cooled to 10 to 15° C. N-chlorosuccinimide (614 g, 4.6 mol) was then added portion-wise, keeping the temperature between 10 and 15° C. The reaction was then warmed to room temperature and stirred for a further 30 minutes. The reaction slurry was then filtered and the organic solution was washed twice with 2M hydrochloric acid (2×1.8 L), followed by two washes with water (2×4.6 L). The organic layer was then dried over anhydrous magnesium sulphate before being concentrated to an oil under vacuum to provide the title compound as a 2:3 mixture of keto:enol tautomers. (780 g, 91%). ¹H NMR (300 MHz, CDCl₃) δ=1.00-1.07(m, 4H), 1.08-1.14(m, 4H), 2.14-2.22(m, 2H-keto), 2.30-2.37(m, 2H-enol), 4.99(s, 1H-keto).

### Preparation 3: 3-Oxobutanoic Acid

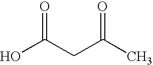

Sodium hydroxide (37.9 g, 947 mmol) was dissolved in water (770 ml) and added to a solution of 3-oxo-butanoic acid methyl ester (100 g, 861 mmol), at room temperature, over 20 minutes. The reaction mixture was stirred for 18 hours, after which time it was quenched with ammonium sulfate (700 g) and acidified slowly with a solution of concentrated hydrochloric acid (21.5 mL) in water (250 mL), with ice cooling. The reaction mixture was then extracted with diethyl ether (6×200 mL) and the combined organic extracts were dried over magnesium sulphate, and concentrated under reduced pressure to provide the title compound (58.2 g, 60%) as a pale yellow oil, which was a mixture of keto:enol tautomers. ¹H NMR (400 MHz, CDCl₃) δ=2.00(s, 3H-enol), 2.30(s, 3H-keto), 3.51 (s, 2H-keto), 5.02(s, 1H-enol).

### Preparation 4: 1-Cyclopropyl-1,3-butanedione

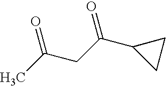

Magnesium turnings (3.04 g, 125 mmol), suspended in methanol (145 mL), were heated to reflux under nitrogen for 1 hour, then cooled to room temperature and the β-keto acid described in Preparation 3 (25.5 g, 250 mmol), dissolved in methanol (25 mL), was added dropwise, with ice-cooling. The reaction mixture was stirred for 1 hour, at room temperature, and then the solvent was removed under reduced pressure to give the magnesium salt of the acid. Meanwhile, cyclopropane-carboxylic acid (9.91 mL, 125 mmol) was dissolved in dimethylformamide (200 mL). Carbonyldiimidazole (22.4 g, 138 mmol) was then added portionwise, under nitrogen, at 0° C. This reaction mixture was stirred for 1.5 hours, and then the magnesium salt from above was added as a solution in N,N-dimethylformamide (100 mL) at 0° C. The reaction mixture was allowed to stir at room temperature for 92 hours, and then it was poured into 2M aqueous hydrochloric acid (85 mL), followed by dilution with water (170 mL). The mixture was extracted with diethyl ether (6×200 mL), and the combined organic extracts were then washed with brine (3×200 mL), dried over magnesium sulphate and concentrated under reduced pressure. The residual orange oil was purified by flash chromatography on silica gel eluting with pentane:diethyl ether (100:0 then 90:10 then 80:20, by volume) to provide the title compound (7.39 g, 24%) as a yellow oil. ¹H NMR (400 MHz, CDCl₃) δ=0.83-0.95(m, 2H), 1.06-1.10(m, 2H), 1.54-1.63(m, 1H), 2.00(s, 3H); LRMS ES+m/z 149 [MNa⁺].

### Preparation 5: 2-Chloro-1-cyclopropyl-1,3-butanedione

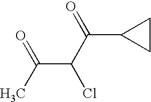

The title compound (7.9 g, 62%, 3:2 mixture of keto:enol tautomers) was prepared by a similar method to that described for Preparation 2, using the diketone described in Preparation 4 as starting material (10 g). ¹H NMR (400 MHz, CDCl₃) δ=1.01-1.04(m, 2H), 1.14-1.20(m, 2H), 2.27(s, 3H), 2.43(m, 1H); LRMS APCI⁺ m/z 161 [MH⁺]; APCI⁻ m/z 159 [M-H]⁻

### Preparation 6: 1-Cyclobutyl-1,3-butanedione

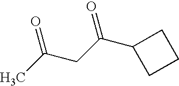

Cyclobutylmethylketone (5.0 g, 50 mmol) was slowly added to a suspension of potassium tert-butoxide (11.4 g, 102 mmol) in tert-butylmethyl ether (100 mL) with cooling in an ice bath. Ethyl acetate (9.8 mL, 102 mmol) was then slowly added and the mixture was stirred in an ice bath for 2 hours before it was carefully quenched onto 2N aqueous hydrochloric acid (250 mL). The organic layer was separated, washed with brine (50 mL) and dried over magnesium sulphate before filtering and concentrating in vacuo to afford the title compound as a pale orange oil (6.43 g, 90%). ¹H NMR (400 MHz, CDCl₃) enol tautomer: δ=1.78-2.00(m, 2H), 2.00(s, 3H), 2.10-2.30(m, 4H), 3.06 (quintet, 1H), 5.41(s, 1H).

### Preparation 7: 2-Chloro-1-cyclobutyl-1,3-butanedione

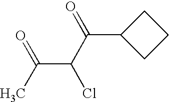

The title compound (4.3 g, 58%, mixture of keto:enol tautomers) was prepared by a similar method to that described for Preparation 2, using the diketone described in Preparation 6 as starting material (6.0 g). ¹H NMR (400 MHz, CDCl₃) 2:3 mixture of keto:enol tautomers: δ=1.80-2.06(m, 2H), 2.18-2.40(m, 6H), 2.20(s, 3H enol), 2.43(s, 3H keto), 3.65(quintet, 1H enol), 3.77(quintet, 1H keto), 4.79(s, 1H keto); LRMS ES⁻ m/z 173, 175 [M-H] chlorine isotopes.

### Preparation 8: 4-(1-Cyclopropanecarbonyl-2-oxo-propoxy)-2,6-dimethyl-benzonitrile

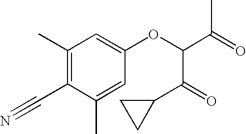

Cesium carbonate (405 g, 1.24 mol) was added to a solution of 4-hydroxy-2,6-dimethyl-benzonitrile (131 g, 0.887 mol) in acetone (2 L). The resulting suspension was stirred at room temperature for 30 minutes. The compound of Preparation 5 (190 g, 1.2 mol) was slowly added and the resulting mixture was stirred at reflux for 2 hours. The reaction was cooled to room temperature and acetic acid (203 mL, 3.6 mol) was carefully added to reaction mixture. The reaction mixture was filtered and concentrated in vacuo. The residue was partitioned between water (1 L) and dichloromethane (3×500 mL) and the combined organic extracts were washed with brine (500 mL), dried over magnesium sulphate, filtered and concentrated in vacuo. The crude material was dissolved in hot isopropanol (3 mUg) and left to cool slowly over 16 hours. The resulting solid was collected by filtration (90 g). The mother liquors were concentrated in vacuo and subjected to flash chromatography (silica gel 1.75 kg eluting with 5% ethyl acetate in pentane) to give a yellow solid. This solid was triturated with pentane to give a white solid which was combined with the solid obtained from the isopropanol crystallisation to afford the title product as a white solid (109 g, 33%). ¹H NMR (400 MHz, CDCl₃) δ=0.85(m, 2H), 1.10(m, 2H), 1.85(m, 1H), 2.00(s, 3H), 2.45(s, 6H), 6.65(s, 2H). LRMS ES⁻ m/z 270 (M-H).

### Preparation 9: 4-(1-Cyclobutanecarbonyl-2-oxo-propoxy)-2,6-dimethyl-benzonitrile

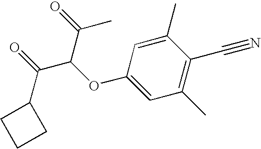

The title compound was prepared in 50% yield by a similar method to that described for Preparation 8, using the chlorodiketone described in Preparation 7 as starting material. ¹H NMR (400 MHz, CDCl₃, enol form) δ=1.75-2.00(m, 2H), 1.95(s, 3H), 2.25-2.40(m, 3H), 2.48-2.52(m, 1H), 2.50(s, 6H), 3.25(quintet, 1H), 6.71 (s, 2H); LCMS Rₜ=3.8 minutes APCI⁺ m/z 286 [MH⁺]; APCI⁻ m/z 284 [M-H]⁻

### Preparation 10: 4-(1-Cyclopropanecarbonyl-2-cyclopropyl-2-oxo-ethoxy)-2,6-dimethyl-benzonitrile

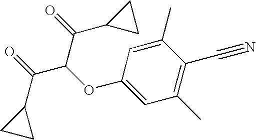

The title compound was prepared in 60% yield by a similar method to that described for Preparation 8, using the chlorodiketone described in Preparation 2 as starting material. ¹H NMR (400 MHz, CDCl₃, enol form) δ=0.87-0.91(m, 4H), 1.12-1.15(m, 4H), 1.79-1.84(m, 2H), 2.52(s, 6H), 6.77(s, 2H); LRMS APCI⁺ m/z 298 [MH⁺]; APCI⁻ m/z 296 [M-H]⁻.

### Preparations 11, 12 and 13

The appropriate diketone of Preparation 8, 9 or 10 was dissolved in acetic acid (2 mL/mmol). Hydrazine hydrate (1.2 equivalents) was then added, and the reaction mixture was stirred at room temperature for 1 hour, under nitrogen. It was then concentrated under reduced pressure, and the residue was purified by flash chromatography on silica gel eluting with dichloromethane:ethyl acetate to provide the title compound as a solid.

### Preparation 11: 4-(5-Cyclopropyl-3-methyl-1H-pyrazol-4-yloxy)-2,6-dimethyl-benzonitrile

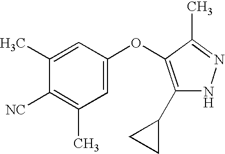

¹H NMR (400 MHz, CDCl₃) δ=0.76-0.82(m, 4H), 1.67(m, 1H), 2.08(s, 3H), 2.47(s, 6H), 6.64(s, 2H); LRMS APCI⁺ m/z 268 [MH⁺]; APCI⁻ m/z 266 [M-H]⁻ ; 62% yield

### Preparation 12: 4-[(3,5-Dicyclopropyl-1H-pyrazol-4-yl)oxy]-2,6-dimethylbenzonitrile

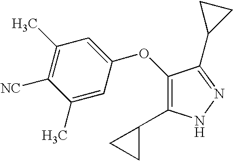

¹H NMR (400 MHz, CDCl₃) δ=0.75-0.81(m, 8H), 1.60-1.66(m, 2H), 2.48(s, 6H), 6.67(s, 2H); LRMS APCI⁺ m/z 294 [MH⁺]; APCI⁻ m/z 292 [M-H]⁻; 68% yield

### Preparation 13: 4-(5-Cyclobutyl-3-methyl-1H-pyrazol-4-yloxy)-2,6-dimethyl-benzonitrile

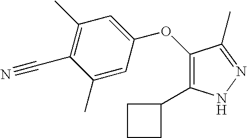

¹H NMR (400 MHz, CDCl₃) δ=1.80-1.88(m, 1H), 1.90-2.00(m, 1H), 2.08(s, 3H), 2.18-2.25(m, 4H), 2.46(s, 6H), 3.39(quintet, 1H), 6.61 (s, 2H); LCMS Rₜ=3.36 minutes APCI⁺ m/z 282(MH+); 57% yield.

### Preparation 14: 4-(2-Cyclopropyl-2-oxoethoxy)-2,6-dimethylbenzonitrile

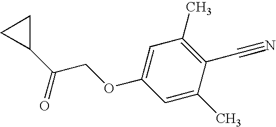

Bromine (12.84 mL, 250 mmol) was added dropwise, over 10 minutes, to an ice-cooled solution of cyclopropylmethylketone (21 g, 250 mmol), in methanol (150 mL), under nitrogen. The reaction was allowed to proceed with the internal temperature being kept under 10° C., until decolourisation of the solution was observed. The reaction mixture was then stirred at room temperature for a further 30 minutes, after which time water (75 mL) was added and the reaction mixture was stirred for a further 15 minutes. The reaction mixture then was diluted with water (225 mL) and extracted 4 times with diethyl ether (50 mL). The organic layers were combined, washed with a 10% aqueous solution of sodium bicarbonate, followed by water, followed by brine, then dried over magnesium sulphate, filtered and concentrated under reduced pressure to provide 2-bromo-1-cyclopropylethanone. Caesium carbonate (30.7 g, 111.16 mmol) was added to a solution of 4-hydroxy-2,6-dimethylbenzonitrile (15.27 g, 101.89 mmol), in acetone (377 mL). Then 2-bromo-1-cyclopropylethanone (15.1 g, 62.6 mmol), in acetone (100 mL), was added dropwise, over 5 minutes, to the resulting suspension and the reaction mixture was heated at reflux for 1.5 hours. It was then concentrated under reduced pressure and the residue was partitioned between an aqueous solution of potassium carbonate and dichloromethane. The organic layer was separated and washed with brine, dried over magnesium sulphate, filtered and then concentrated under reduced pressure. The crude product was purified by flash chromatography on silica gel eluting with dichloromethane:pentane (50:50 to 80:20, by volume) to provide the title compound (13.5 g, 64%) as a solid. ¹H-NMR (400 MHz, CDCl₃): δ=0.97-1.01(m, 2H), 1.12-1.15(m, 2H), 2.19(m, 1H), 2.47(s, 6H), 4.71(s, 2H), 6.61(s, 2H); LRMS: APCI⁺: m/z 230 [MH⁺]

### Preparation 15: 4-{[(E/Z)-1-(Cyclopropylcarbonyl)-2-(dimethylamino)vinyl]oxy}-2,6-dimethylbenzonitrile

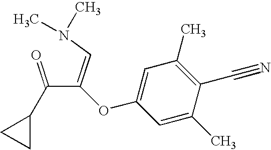

The benzonitrile of Preparation 14 (11.8 g, 51.46 mmol) and N,N-dimethylformamide dimethyl acetal (13.7 mL, 102.93 mmol) were heated at 105° C. for 12 hours. The reaction mixture was then concentrated under reduced pressure. The crude product was purified by flash chromatography on silica gel eluting with dichloromethane:pentane (50:50 then 80:20 then 100:0, by volume) to provide the title compound (11.19 g, 76%) as a white solid. ¹H-NMR (400 MHz, CDCl₃): δ=0.63(brs, 2H), 0.91(brs, 2H), 1.93(m, 1H), 2.44(s, 6H), 2.96(s, 6H), 6.69(s, 2H); LRMS: APCI⁺: m/z 285 [MH⁺].

### Preparation 16: N′-(1,1-Dioxo-1-λ⁶-thietan-3-yl)-hydrazinecarboxylic acid tert-butyl ester

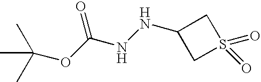

A mixture of 3-thietanone-1,1-dioxide (Tetrahedron Lett., 1963, 1297) (3.0 g, 24.97 mmol) and hydrazinecarboxylic acid tert-butylester (3.14 g, 23.7 mmol) was heated at reflux in toluene (120 mL) for 16 hours in a Dean-Stark apparatus. The reaction mixture was evaporated in vacuo. The residue was suspended in ethanol (mL) and sodium borohydride was added. After 24 hours water (150 mL) was added to the reaction mixture which was then extracted with ethylacetate, (3×200 mL). The organic extracts were combined, washed with brine (250 mL), dried over magnesium sulfate and evaporated to dryness to afford the title compound as a white solid (4.69 g, 79%). ¹H-NMR (400 MHz, CDCl₃): δ=1.35(s, 9H), 3.75-4.88(m, 3H), 4.17-4.25(m, 2H), 5.19(s, 1H), 8.43(s, 1H); LRMS: APCI⁺: m/z 235 [MH⁺], 181 [M-^{t}Bu].

### Preparation 17: (1,1-Dioxo-1-λ⁶-thietan-3-yl)-hydrazine

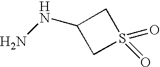

Amberlyst 15 resin was purified and quantified as described in J. Org. Chem., 1998 63(10) 3471 to afford a resin with acid loading capacity of 3.75 mmol/g. This purified resin (15 g) was added to a solution of the title compound of preparation 16 (4.69 g, 19.85 mmol) in dichloromethane (200 mL) and the mixture stirred at room temperature for 24 hours. An additional aliquot of amberlyst 15 resin (20 g) was added and the reaction stirred for another 24 hours. The resin was removed by filtration washed with dichloromethane (100 mL) and pentane (100 mL). Then the resin was transferred to a round bottom flask and ammonia solution (300 mL, 2M in methanol, 600 mmol) was added. The mixture was stirred for 30 minutes. The resin was removed by filtration and the filtrate retained. The resin was suspended again in ammonia solution (300 mL, 2M in methanol, 600 mmol) and stirred for 30 minutes. The resin was removed by filtration and washed with methanol (200 mL). The filtrates were combined and evaporated to dryness to afford the title compound as a gum (1.93 g, 71%). ¹H-NMR (400 MHz, d₆-DMSO): δ=2.42-2.43(m, 1H), 3.50-3.76(m, 1H), 3.80-3.92(m, 2H), 4.03-4.14(m, 2H), 4.30(brs, 3H).

### EXAMPLE 1

### 4-(3,5-Dicyclopropyl-1-oxetan-3-yl-1H-pyrazol-4-yloxy)-2,6-dimethyl-benzonitrile

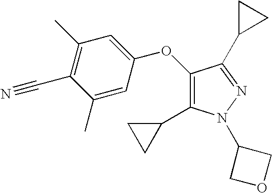

Sodium Hydride (60% dispersion in mineral oil, 16.4 mg, 0.41 mmol) was added to a solution of the compound obtained from Preparation 12 (100 mg, 0.341 mmol) in tetrahydrofuran (0.75 mL). This mixture was heated to 50° C. A solution of toluene-4-sulfonic acid oxetan-3-yl-ester (93.4 mg, 0.41 mmol) in tetrahydrofuran (0.75 mL) was then added, and this new mixture was stirred at 50° C. for 12 hours. This mixture was partitioned between water (5 mL) and diethyl ether (2×5 mL). The combined organics were washed with saturated brine (5 mL) dried over magnesium sulphate filtered and evaporated under reduced pressure. This residue was purified by purified by preparative high performance liquid chromatography to give the title compound as an off white gum (5 mg, 4%). ¹H NMR (400 MHz, CDCl₃): δ 0.55-1.70(m, 10H), 2.45(m, 6H), 4.90(m, 2H), 5.20(m, 2H), 5.60(m, 1H), 6.60(s, 2H); APCI MS m/z 294 [M-CH(CH₂)₂O]⁺.

### EXAMPLE 2

### 4-[3,5-Dicyclopropyl-1-(1,1-dioxo-thietan-3-yl)-1H-pyrazol-4-yloxy]-2,6-dimethyl-benzonitrile

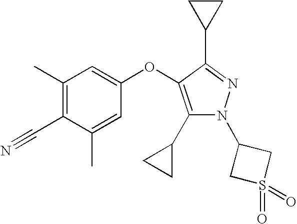

Potassium tert-butoxide (18 mg, 0.16 mmol) was added to a solution of the compound of Preparation 12 (40 mg, 0.14 mmol) in N-methylpyrrolidinone (0.6 mL) and the mixture stirred at room temperature for 10 minutes. 3-Chloro-thietane-1,1-dioxide (25 mg, 0.18 mmol) was added and the resulting mixture was subjected to microwave irradiation, heating at 150° C. for 40 minutes. The reaction mixture was poured into water (20 mL) and the resulting white precipitate collected by filtration, washed with water (10 mL) and dried under high vacuum. Trituration with hot ethanol (1 mL) afforded the title compound as an off white solid (35 mg, 65%). ¹H NMR (400 MHz, CDCl₃) δ=0.60-0.90(m, 8H), 1.39-1.55(m, 2H), 2.46(s, 6H), 4.38-4.45(m, 2H), 4.84-4.89(m, 2H), 5.20-5.30(m, 1H), 6.59(s, 2H); LCMS Rₜ=3.4 mins, ES⁺ m/z=398 (MH+).

### EXAMPLE 3

### 4-[3,5-Dicyclopropyl-1-(tetrahydro-furan-3-yl)-1 H-pyrazol-4-yloxy]-2,6-dimethyl-benzonitrile

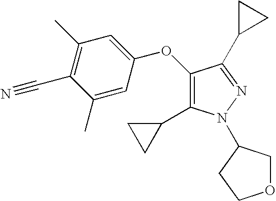

(Tetrahydro-furan-3-yl)-hydrazine (118 mg, 0.67 mmol) was added to a solution of the compound of Preparation 10 (100 mg, 0.34 mmol) in methanol (3.5 mL). Hydrochloric acid (0.67 mL, 2M in diethylether, 1.35 mmol) was added and the reaction heated to 60° C. for 16 hours. The reaction mixture was concentrated in vacuo and the residue purified by automated preparative HPCL to afford the title compound. LRMS APCI m/z=364 (MH⁺) ESI m/z=364 (MH⁺).

### EXAMPLE 4

### 4-[3,5-Dicyclopropyl-1-(tetrahydro-thiopyran-4-yl)-1H-pyrazol-4-yloxy]-2,6-dimethyl-benzonitrile

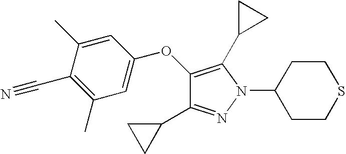

A solution of (tetrahydro-thiopyran-4-yl)-hydrazine (182 mg, 0.74 mmol) in methanol (2 mL) was added to a solution of the compound obtained from Preparation 10 (200 mg, 0.673 mmol) in methanol (5 mL). This mixture was stirred at 70° C. for 16 hours. The reaction mixture was concentrated under reduced pressure. This residue was partitioned between saturated sodium hydrogen carbonate (10 mL) and diethyl ether (2×10 mL). The combined organic layers were washed with saturated brine (10 mL) dried over magnesium sulphate filtered and evaporated under reduced pressure to give a pale yellow oil. This residue was purified by column chromatography eluting with 7% ethyl acetate in pentane to give the title compound as a pale yellow oil (120 mg, 45%). ¹H NMR (400 MHz, CDCl₃) δ=0.60-0.90(m, 8H), 1.45(m, 1H), 1.55(m, 1H), 2.20(m, 2H), 2.40(m, 2H), 2.45(s, 6H), 2.80(m, 4H), 4.25(m, 1H), 6.60(s, 2H); LRMS APCI m/z 394 [M+H]⁺.

### EXAMPLE 5

### 4-[3,5-Dicyclopropyl-1-(1,1-dioxo-hexahydro-1 λ⁶-thiopyran-4-yl)-1H-pyrazol-4-yloxy]-2,6-dimethyl-benzonitrile

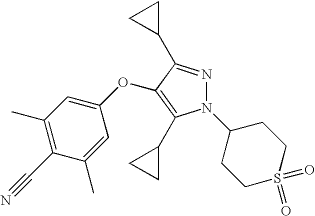

Oxone (125 mg, 0.20 mmol) was added in one portion to a solution of the compound obtained from Example 2 (80 mg, 0.20 mmol) in Methanol (6 mL) and water (1.2 mL). The reaction mixture was stirred at room temperature for 16 hours before concentrating under reduced pressure. This residue was partitioned between water (10 mL) and diethyl ether (2×10 mL). The combined organics were washed with saturated brine (10 mL) dried over magnesium sulphate filtered and evaporated under reduced pressure. This residue was purified by column chromatography eluting with 20% ethyl acetate in pentane to give the title compound as a white solid (62 mg, 72%). ¹H NMR (400 MHz, CDCl₃) δ=0.60-1.60(m, 10H), 2.45(m, 8H), 2.60(m, 2H), 3.00(m, 2H), 3.65(m, 2H), 4.60(m, 1H), 6.60(s, 2H); LRMS ES⁺ m/z 426 [M+H]⁺.

### EXAMPLE 6

### 4-[3,5-Dicyclopropyl-1-(tetrahydro-pyran-4-yl)-1H-pyrazol-4-yloxy]-2,6-dimethyl-benzonitrile

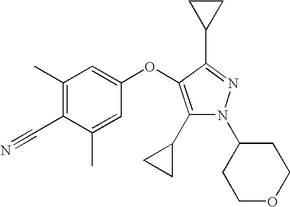

Using a procedure similar to Example 4, but using (tetrahydro-pyran-4-yl)-hydrazine. The title compound was prepared as a colourless gum. (106 mg, 84%). ¹H NMR (400 MHz, CDCl₃) δ=0.60-0.90(m, 8H), 1.40-1.60(m, 2H), 1.85(m, 2H), 2.35(m, 2H), 2.45(s, 6H), 3.55(m, 2H), 4.15(m, 2H), 4.45(m, 1H), 6.60(s, 2H); LRMS APCI m/z 378 [M+H]⁺.

### EXAMPLE 7

### rac-4-[3,5-Dicyclopropyl-1-(1,1-dioxo-tetrahydro-1λ⁶-thiophen-3-yl)-1H-pyrazol-4-yloxy]-2,6-dimethyl-benzonitrile

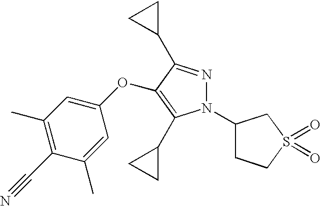

(1,1-Dioxo-tetrahydro-1λ⁶-thiophen-3-yl)-hydrazine hydrochloride (167 mg, 1.11 mmol) was added to a stirred solution of the compound of Preparation 10 (300 mg, 1.01 mmol) in methanol (20 mL) at room temperature. Hydrochloric acid (1.88 mL, 2M in diethyl ether, 3.75 mmol) was added and the resulting mixture heated to 60° C. for 16 hours. The mixture was concentrated under reduced pressure and the residues partitioned between diethyl ether (100 mL) and saturated sodium hydrogen carbonate solution (100 mL). The organic layer was separated and further washed with water (50 mL), brine (50 mL), dried over magnesium sulphate and concentrated in vacuo. The residue was purified by flash chromatography (eluting with dichloromethane) to afford the title compound as a solid (250 mg, 60%). ¹H NMR (400 MHz, CDCl₃) δ=0.67-0.72(m, 2H), 0.76-0.84(m, 4H), 0.84-0.90(m, 2H), 1.45-1.55(m, 2H), 2.48(s, 6H), 2.58-2.80(m, 2H), 3.15-3.25(m, 1H), 3.46(dd, 1H), 3.53-3.60(m, 1H), 3.63(dd, 1H), 5.25(quintet, 1H), 6.61 (s, 2H); LCMS Rₜ=3.50 mins ES+, m/z=412 (MH+).

### EXAMPLE 8

### Enantiomer 1-4-[3,5-Dicyclopropyl-1-(1,1-dioxo-tetrahydro-1λ⁶-thiophen-3-yl)-1H-pyrazol-4-yloxy]-2,6-dimethyl-benzonitrile

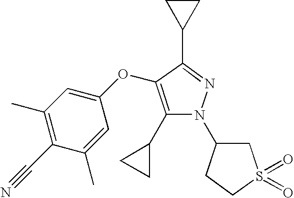

and

### EXAMPLE 9

### Enantiomer 2-4-[3,5-Dicyclopropyl-1-(1,1-dioxo-tetrahydro-1λ^{6*}-thiophen-3-yl)-1H-pyrazol-4-yloxy]-2,6-dimethyl-benzonitrile

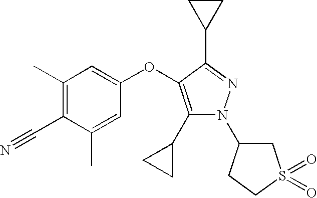

The compound of Example 7 (250 mg) was separated into it's constitutive enantiomers using chiral HPLC on a Chiralcel OD-H (250*21.2 mm i.d.) column using ethanol as the eluant at a flow rate of 12 mL/minute to afford Example 8 the first eluting enantiomer (74 mg) and Example 9 the second eluting enantiomer (70 mg).

### EXAMPLE 8

### Enantiomer 1

¹H NMR (400 MHz, CDCl₃) 0.67-0.72(m, 2H), 0.76-0.84(m, 4H), 0.84-0.90(m, 2H), 1.45-1.55(m, 2H), 2.48(s, 6H),2.58-2.80(m, 2H), 3.15-3.25(m, 1H), 3.46(dd, 1H), 3.53-3.60(m, 1H), 3.63(dd, 1H), 5.25(quintet, 1H), 6.61(s, 2H); LCMS Rₜ=3.50 mins ES+, m/z=412 (MH+). Optical rotation specific rotation of −62.25° at 25° C., 2 mg/mL in chloroform, path length 100 mm, wavelength 365 nm.

### EXAMPLE 9

### Enantiomer 2

¹H NMR (400 MHz, CDCl₃) 0.67-0.72(m, 2H), 0.76-0.84(m, 4H), 0.84-0.90(m, 2H), 1.45-1.55(m, 2H), 2.48(s, 6H),2.58-2.80(m, 2H), 3.15-3.25(m, 1H), 3.46(dd, 1H), 3.53-3.60(m, 1H), 3.63(dd, 1H), 5.25(quintet, 1H), 6.61(s, 2H); LCMS Rₜ=3.50 mins ES+, m/z=412 (MH+). Optical rotation specific rotation of +56.63° at 25° C., 2 mg/mL in chloroform, path length 100 mm, wavelength 365 nm.

### EXAMPLE 10

### 4-(3-Cyclopropyl-5-methyl-1-(1,1-dioxo-thietan-3-yl)-1H-pyrazol-4-yloxy)-2,6-dimethyl-benzonitrile

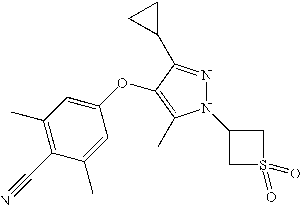

and

### EXAMPLE 11

### 4-(3-Methyl-5-cyclopropyl-1-(1,1-dioxo-thietan-3-yl)-1H-pyrazol-4-yloxy)-2,6-dimethyl-benzonitrile

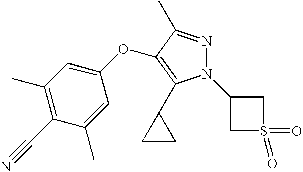

The compound of Preparation 11 (100 mg, 0.37 mmol) was first treated with potassium tert-butoxide (50 mg, 0.45 mmol) in N-methylpyrrolidinone (0.6 mL) for 15 minutes before 3-chloro-thietane 1,1-dioxide (68 mg, 0.49 mmol) was added and the reaction mixture was subjected to microwave heating at 150° C. for 40 minutes. The reaction mixture was poured into water (50 mL) and the resulting off-white precipitate was collected by filtration, washed again with water (2×10 mL) and dried under air flow. The two regioisomers were separated by preparative HPLC (Phenomenex C8(2) 5 μm, mobile phase A: 0.1% formic acid in water, mobile phase B: 0.1% formic acid in acetonitrile, flow rate 20 mL/min, detection at 225 nm and 254 nm, room temperature, mobile phase B gradient: 50% to 80% over 5.5 minutes and up to 100% over 16 minutes total time yielding 23 mg, 17% of the compound of Example 10 4-[3-Cyclopropyl-1-(1,1-dioxo-1λ⁶-thietan-3-yl)-5-methyl-1H-pyrazol-4-yloxy]-2,6-dimethyl-benzonitrile. ¹H NMR (400 MHz, CDCl₃) δ=0.74-0.84(m, 4H), 1.54-1.60(m, 1H), 2.05(s, 3H), 2.45(s, 6H), 4.36-4.43(m, 2H), 4.84-4.98(m, 3H), 5.24-5.33(m, 1H), 6.60(s, 2H); LCMS Rₜ=3.27 mins, ES⁺ m/z=372 (MH+) and 12 mg, 9% of the compound of Example 11 4-[5-Cyclopropyl-1-(1,1-dioxo-1λ⁶-thietan-3-yl)-3-ethyl-1H-pyrazol-4-yloxy]-2,6-dimethyl-benzonitrile. ¹H NMR (400 MHz, CDCl₃) δ=0.62-0.66(m, 2H), 0.83-0.88(m, 2H), 1.42-1.49(m, 1H), 1.99(s, 3H), 2.45(s, 6H), 4.43-4.49(m, 2H), 4.86-4.92(m, 2H), 5.24-5.33(m, 1H), 6.53(s, 2H); LCMS Rₜ=3.23 mins, ES⁺ m/z=372 (MH+).

### EXAMPLE 12

### rac-4-[5-Cyclobutyl-1-(1,1-dioxo-tetrahydro-1λ⁶-thiophen-3-yl)-3-methyl-1H-pyrazol-4-yloxy]-2,6-dimethyl-benzonitrile

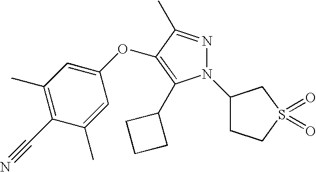

and

### EXAMPLE 13

### rac-4-[3-Cyclobutyl-1-(1,1-dioxo-tetrahydro-1λ⁶-thiophen-3-yl)-5-methyl-1H-pyrazol-4-yloxy]-2,6-dimethyl-benzonitrile

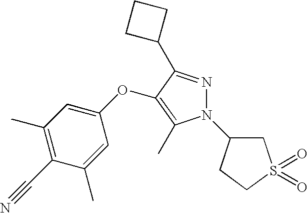

(1,1-Dioxo-tetrahydro-1λ⁶-thiophen-3-yl)-hydrazine hydrochloride (174 mg, 1.16 mmol) was added to a stirred solution of the compound of Preparation 9 (300 mg, 1.05 mmol) in methanol (10 mL) at room temperature. Hydrochloric acid (3 mL, 2M in diethylether, 6 mmol) was added and the resulting mixture heated to 60° C. for 16 hours. The mixture was concentrated under reduced pressure and the residues partitioned between diethyl ether (50 mL) and saturated sodium hydrogen carbonate solution (50 mL). The organic layer was separated and further washed with water (25 mL), brine (25 mL), dried over magnesium sulphate and concentrated under reduced pressure. The residue was purified by flash chromatography (eluting with 20% ethyl acetate in hexane). Subsequently the regioisomers were separated by preparative high-performance liquid chromatography (Phenomenex C8(2) 5 μm, mobile phase A: 0.1% formic acid in water, mobile phase B: 0.1% formic acid in acetonitrile, flow rate 20 mL/min, detection at 225 nm and 254 nm, room temperature, mobile phase B gradient: 50% to 80% over 5.5 minutes and up to 100% over 16 minutes total time yielding 15 mg, 4% of the compound of Example 12 rac-4-[5-Cyclobutyl-1-(1,1-dioxo-tetrahydro-1λ⁶-thiophen-3-yl)-3-methyl-1H-pyrazol-4-yloxy]-2,6-dimethyl-benzonitrile. ¹H NMR (400 MHz, CDCl₃) δ=1.78-1.88(m, 1H), 1.95-2.05(m, 1H), 1.99(s, 3H), 2.17-2.34(m, 3H), 2.49(s, 6H) 2.55-2.65(m, 1H), 2.70-2.80(m, 1H), 3.18(dt, 1H), 3.38-3.48(m, 2H), 3.60(dt, 1H), 3.64(dd, 2H), 4.87(quintet, 1H), 6.59(s, 2H); LRMS ES⁺ m/z 400(MH+). and 7 mg, 2% of the compound of Example 13: rac-4-[3-Cyclobutyl-1-(1,1-dioxo-tetrahydro-1λ⁶-thiophen-3-yl)-5-methyl-1H-pyrazol-4-yloxy]-2,6-dimethyl-benzonitrile. ¹H NMR (400 MHz, CDCl₃) δ=1.63-1.82(m, 2H), 1.96(s, 3H), 1.95-2.02(m, 2H), 2.04-2.13(m, 2H), 2.34(s, 6H) 2.45-2.55(m, 1H), 2.60-2.70(m, 1H), 3.03-3.17(m, 2H), 3.34(dd, 1H), 3.48(dt, 1H), 3.60(dd, 1H), 4.81 (quintet, 1H), 6.42(s, 2H); LRMS ES⁺ m/z 400(MH+).

### EXAMPLE 14

### rac-4-[5-Cyclopropyl-1-(1,1-dioxo-tetrahydro-1λ⁶-thiophen-3-yl)-3-methyl-1H-pyrazol-4-yloxy]-2,6-dimethyl-benzonitrile

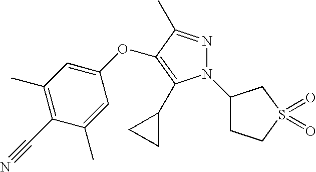

and

### EXAMPLE 15

### rac-4-[3-Cyclopropyl-1-(1,1-dioxo-tetrahydro-1λ⁶-thiophen-3-yl)-5-methyl-1H-pyrazol-4-yloxy]-2,6-dimethyl-benzonitrile

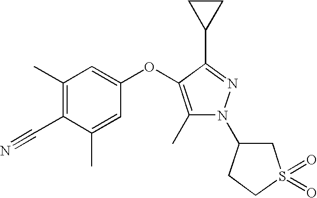

(1,1-Dioxo-tetrahydro-1λ⁶-thiophen-3-yl)-hydrazine hydrochloride (249 mg, 1.66 mmol) was added to a stirred solution of the compound of Preparation 8 (300 mg, 1.11 mmol) in methanol (10 mL) at room temperature. Hydrochloric acid (2 mL, 2M in diethylether, 4 mmol) was added and the resulting mixture heated to 60° C. for 16 hours. The mixture was concentrated under reduced pressure and the residues partitioned between ethyl acetate (50 mL) and water (50 mL). The organic layer was separated and further washed with brine (25 mL), dried over magnesium sulphate and concentrated under reduced pressure. The residue was purified by flash chromatography (eluting with 10% isopropanol in toluene). The product containing fractions were evaporated to dryness and the resulting solid recrystallised from isopropanol (20 mL) and water (10 mL) to afford the title compound of Example 14 as a white solid (100 mg, 24%). The mother liquors were evaporated and the residue was purified by preparative HPLC using a Phenomenex C8(2) 5 μm (150×10 mm i.d.) and a mobile phase of 0.6 minutes elution with 5% of acetonitrile in water containing 0.1% of formic acid followed by gradient elution until after 8 minutes the elution solvent is 100% acetonitrile containing 0.1% formic acid, this elution solvent is continued for a further minute. Preparative HPLC purification afforded (10 mg, 2.4%) of Example 15.

### EXAMPLE 14

### rac-4-[5-Cyclopropyl-1-(1,1-dioxo-tetrahydro-1 λ⁶-thiophen-3-yl)-3-methyl-1H-pyrazol-4-yloxy]-2,6-dimethyl-benzonitrile

¹H NMR (400 MHz, CDCl₃) δ=0.65-0.70(m, 2H), 0.80-0.85(m, 2H), 1.48-1.52(m, 1H), 1.98(s, 3H), 2.43(s, 6H) 2.59-2.80(m, 2H), 3.19(dt, 1H), 3.44(dd, 1H), 3.59(dd, 1H), 3.61(dd, 1H), 3.64 (dd, 2H), 5.22 (quintet, 1H), 6.53 (s, 2H); LCMS Rₜ=3.30 mins, APCI m/z 386 (MH+).

### EXAMPLE 15

### rac-4-[3-Cyclopropyl-1-(1,1-dioxo-tetrahydro-1λ⁶-thiophen-3-yl)-5-methyl-1H-pyrazol-4-yloxy]-2,6-dimethyl-benzonitrile

¹H NMR (400 MHz, CDCl₃) δ=0.73-0.81(m, 4H), 1.52-1.62(m, 1H), 2.10(s, 3H), 2.49(s, 6H) 2.55-2.65(m, 1H), 2.69-2.78(m, 1H), 3.18(dt, 1H), 3.44(dd, 1H), 3.48(dt, 1H), 3.58(dt, 1H), 3.65(dd, 1H), 4.92(quintet, 1H), 6.63(s, 2H); LCMS Rₜ=3.18 mins, APCI m/z 386(MH+).

### EXAMPLE 16

### 4-(3-Cyclopropyl-5-methyl-1-(1,1-dioxo-1λ⁶-thietan-3-yl)-1H-pyrazol-4-yloxy)-2-methyl-benzonitrile

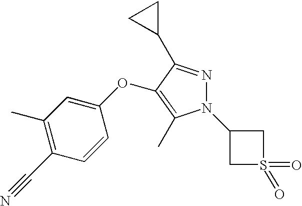

The title compound of Example 16 may be prepared using analogous methods to the synthesis of Example 10 but employing 2-methylbenzonitrile in place of 2,6-dimethylbenzonitrile in Preparation 8.

### EXAMPLE 17

### 4-(3-Cyclopropyl-5-methyl-1-(1,1-dioxo-1λ⁶-thietan-3-yl)-1H-pyrazol-4-yloxy)-2-chloro-benzonitrile

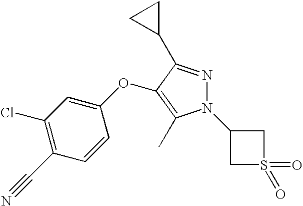

The title compound of Example 17 may be prepared using analogous methods to the synthesis of Example 10 but employing 2-chlorobenzonitrile in place of 2,6-dimethylbenzonitrile in Preparation 8.

### EXAMPLE 18

### 4-[5-Cyclopropyl-1-(1,1-dioxo-1λ⁶-thietan-3-yl)-1H-pyrazol-4-yloxy]-2,6-dimethyl-benzonitrile and Example 19: 4-[3-Cyclopropyl-1-(1,1-dioxo-1λ⁶-thietan-3-yl)-1H-pyrazol-4-yloxy]-2,6-

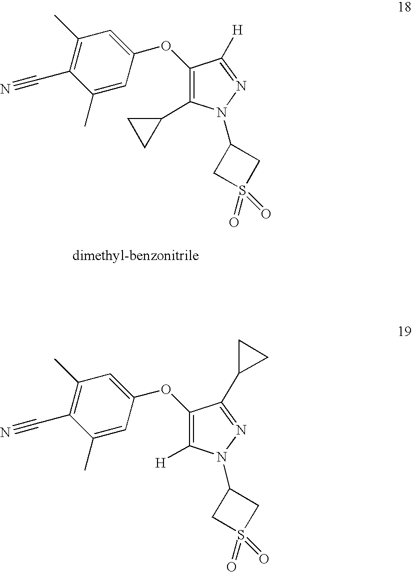

A solution of the hydrazine of Preparation 17 (1.98 g, 11.6 mmol) in acetic acid (2 mL) was added to a suspension of the compound of Preparation 15 (2.0 g, 7.03 mmol) in acetic acid (20 mL). The reaction mixture was stirred at room temperature for 16 hours before evaporating in vacuo. The residue was partitioned between water (200 mL) and ethylacetate (200 mL), the organic phase was further washed with sodium bicarbonate solution (100 mL sat. aqueous), brine (100 mL), dried (magnesium sulphate) and concentrated under vacuum to afford the title compounds as a yellow solid as a 3:1 mixture of Example 18: Example 19 (2.57 g, 100%). ¹H-NMR (400 MHz, CDCl₃) δ=0.70-0.76(m, 2Hₘₐⱼₒᵣ), 0.79-0.88(m, 4Hₘᵢₙₒᵣ), 0.90-0.95(m, 2Hₘₐⱼₒᵣ), 1.46-1.57(m, 1Hₘₐⱼₒᵣ), 1.60-1.68(m, 1Hₘᵢₙₒᵣ), 2.48(s, 6Hₘₐⱼₒᵣ), 2.49(s, 6Hₘᵢₙₒᵣ), 4.50-4.60(m, 3Hₘₐⱼₒᵣ), 4.68-4.76(m, 2Hₘᵢₙₒᵣ), 4.88-4.96(m, 2Hₘₐⱼₒᵣ), 4.98-5.04(m, 1Hₘᵢₙₒᵣ), 5.31-5.42(m, 2Hₘᵢₙₒᵣ), 6.62(s, 2Hₘₐⱼₒᵣ), 6.68(s, 2Hₘᵢₙₒᵣ), 7.38(s, 1Hₘᵢₙₒᵣ), 7.39(s, 1Hₘₐⱼₒᵣ). LRMS APCI: m/z 358 [MH]^{+.}

### EXAMPLE 20

### 4-[5-Chloro-3-cyclopropyl-1-(1,1-dioxo-1λ⁶-thietan-3-yl)-1H-pyrazol-4-yloxy]-2,6-dimethyl-benzonitrile

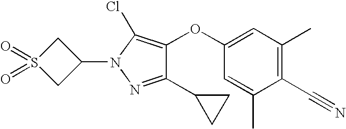

and

### EXAMPLE 21

### 4-[3-Chloro-5-cyclopropyl-1-(1,1-dioxo-1λ⁶-thietan-3-yl)-1H-pyrazol-4-yloxy]-2,6-dimethyl-benzonitrile

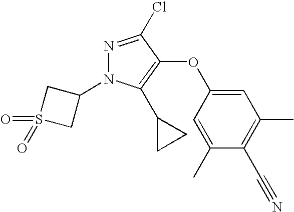

N-Chlorosuccinimide (224 mg, 1.68 mmol) was added to a solution of the 3:1 mixture of Examples 18 and 19 (500 mg, 1.40 mmol) in acetonitrile (10 mL) and the mixture stirred at 60° C. After 4 hours a further aliquot of N-chlorosuccinimide (150 mg, 1.13 mmol) was added and the reaction stirred for a further 35 minutes at 60° C. The reaction mixture was evaporated in vacuo to afford a brown solid which was purified by silica chromatography (50 g column, eluting with 10% ethylacetate in toluene) to afford the title compound of Example 20 (first eluting product) as a white solid (79 mg) and the title compound of Example 21 (second eluting product) as a white solid (38 mg).

### EXAMPLE 20

¹H-NMR (400 MHz, CDCl₃): δ 0.81-0.91(m, 4H), 1.65(m, 1H), 2.49(s, 6H), 4.48(m, 2H), 4.85(m, 2H), 5.18(m, 1H), 6.66(s, 2H). LCMS Rₜ=3.33 mins, ES⁺ m/z=392 (MH+).

### EXAMPLE 21

¹H-NMR (400 MHz, CDCl₃): δ 0.70-0.78(m, 2H), 0.90-0.96(m, 2H), 1.50(m, 1H), 2.49(s, 6H), 4.49(m, 2H), 4.88(m, 2H), 5.33(m, 1H), 6.58(s, 2H). LCMS Rₜ=3.28 mins, ES⁺ m/z=392 (MH+).

Biological Data (Progesterone receptor IC₅₀ (nM))

| | | Ex- |
|---|---|---|
| | PR | ample |
| Structure | IC50 | No. |
| 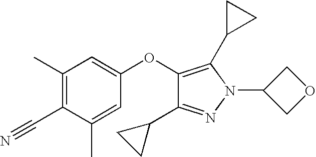 | 82.6nM | Ex-ample 1 |
| 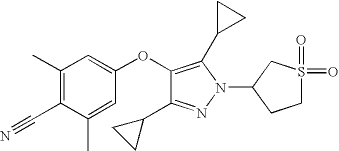 | 13.8nM | Ex-ample 7 |
| 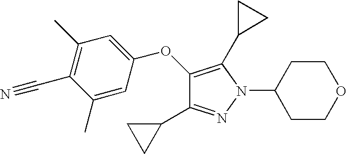 | 56.3nM | Ex-ample 6 |
| 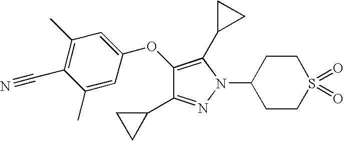 | 91.3nM | Ex-ample 5 |
| 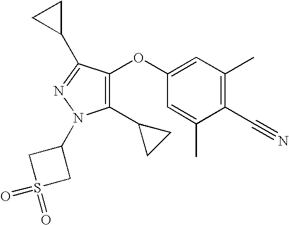 | 14.7nM | Ex-ample 2 |
| 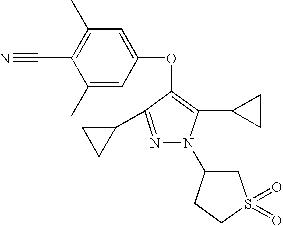 | 33.8nM | Ex-ample 8 |
| 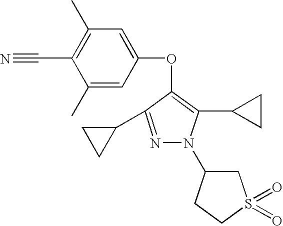 | 25.9nM | Ex-ample 9 |
| 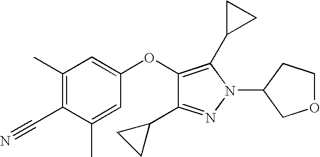 | 67.4nM | Ex-ample 3 |
| 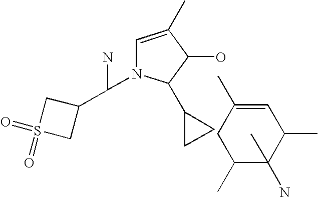 | 50.4nM | Ex-ample11 |
| 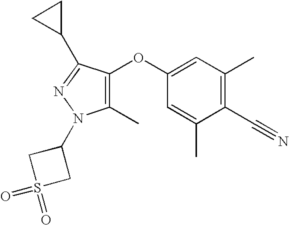 | 27.6nM | Ex-ample10 |
| 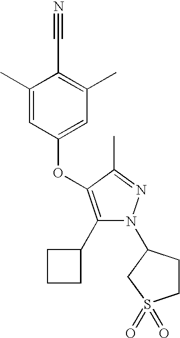 | 1490nM | Ex-ample12 |
| 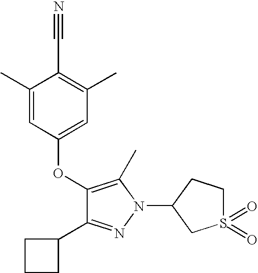 | 202nM | Ex-ample13 |
| 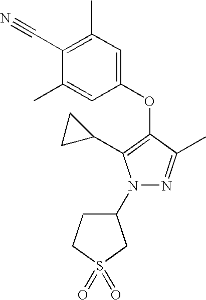 | 133nM | Ex-ample14 |
| 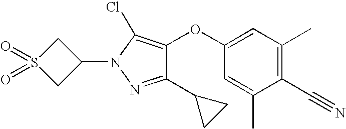 | 22nM | Ex-ample20 |
| 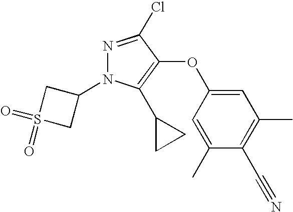 | 108nM | Ex-ample21 |

1. A compound of Formula (I) 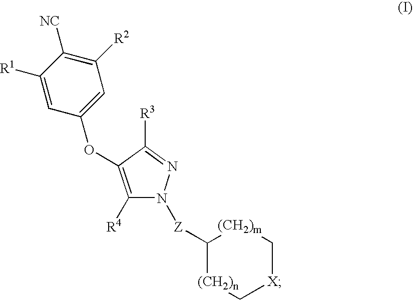
or a pharmaceutically acceptable salt, solvate (including hydrate) of said compounds and salt, or a prodrug of said compound, or a pharmaceutically acceptable salt or solvate of said prodrug, wherein:
R¹ and R² are independently selected from H, C₁₋₆ alkyl optionally substituted by one or more halogen, C₁₋₆alkyloxy optionally substituted by one or more halogen, CN, and halo;
R³ is selected from the group consisting of H; C₁₋₆alkyl; C₁₋₆alkyloxy; C₃₋₈cycloalkyl; and halo;
R⁴ is C₁₋₆ alkyl optionally substituted by one or more fluorine and/or nitrile groups, C₁₋₆alkyloxy, C₃₋₈cycloalkyl optionally substituted by one or more fluorine and/or nitrile groups, or halo;
X represents O, S, S(O) or SO₂;
Z represents a bond or (CR⁵R⁶); where R⁵ and R⁶ are independently selected from the group consisting of H and C₁₋₆alkyl; or R⁵ and R⁶, together with the carbon to which they are attached, form a 3 to 6 membered carbocyclic ring which optionally bears from 1 to 3 substituents independently selected from the group consisting of halo; cyano; hydroxyl; C₁₋₄alkyl; C₁₋₄haloalkyl; C₁₋₄haloalkyloxy and C₁₋₄alkyloxy; and
m and n independently are 0, 1 or 2 provided that m+n is not more than 3;
and wherein said alkyl, cycloalkyl or alkoxy groups are independently optionally substituted with from 1 to 5 substituents independently selected from the group consisting of halo, cyano, hydroxyl, C₁₋₄alkyl; C₁₋₄haloalkyl; C₁₋₄haloalkyloxy and C₁₋₄alkyloxy.

2. A compound, salt, solvate or prodrug according to claim 1 wherein R¹ and R² are independently H, Cl, C₁₋₃ alkyl, optionally substituted by one or more halogen, or C₁₋₃ alkyloxy, optionally substituted by one or more halogen.

3. A compound, salt, solvate or prodrug according to claim 1 wherein R³ is methyl, ethyl, cyclopropyl, or chloro.

4. A compound, salt, solvate or prodrug according to claim 1 wherein R⁴ is C₁₋₆ alkyl or C₃₋₈ cycloalkyl.

5. A compound, salt, solvate or prodrug according to claim 1 wherein Z is a bond or (CH₂).

6. A compound, salt, solvate or prodrug according to claim 1 wherein m+n is zero, or m is 1 and n is 1.

7. A compound, salt, solvate or prodrug according to claim 1 wherein R¹ and R² are independently H, Cl, CH₃, CF₃, OCF₃ or OCH₃.

8. A compound, salt, solvate or prodrug according to claim 1 wherein R³ is methyl or cyclopropyl.

9. A compound, salt, solvate or prodrug according to claim 1 wherein R⁴ is C₁₋₄ alkyl or C₃₋₄ cycloalkyl.

10. A compound, salt, solvate or prodrug according to claim 1 wherein Z is a bond.

11. A compound, salt, solvate or prodrug according to claim 1 wherein X is O or SO₂.

12. A compound, salt, solvate or prodrug according to claim 1 wherein R¹ or R² is CH₃.

13. A compound, salt, solvate or prodrug according to claim 1 wherein R⁴ is methyl, isopropyl, cyclopropyl or cyclobutyl.

14. A compound, salt, solvate or prodrug according to claim 13 wherein R⁴ is cyclopropyl.

15. A compound, salt, solvate or prodrug according to claim 1 wherein X is SO₂.

16. A compound according to claim 1 selected from the group consisting of:
4-(3,5-dicyclopropyl-1-oxetan-3-yl-1H-pyrazol-4-yloxy)-2,6-dimethyl-benzonitrile;
4-[3,5-dicyclopropyl-1-(1,1-dioxo-thietan-3-yl)-1H-pyrazol-4-yloxy]-2,6-dimethyl-benzonitrile;
4-[3,5-dicyclopropyl-1-(tetrahydro-furan-3-yl)-1H-pyrazol-4-yloxy]-2,6-dimethyl-benzonitrile;
4-[3,5-dicyclopropyl-1-(tetrahydro-thiopyran-4-yl)-1H-pyrazol-4-yloxy]-2,6-dimethyl-benzonitrile;
4-[3,5-dicyclopropyl-1-(1,1-dioxo-hexahydro-1 λ⁶-th iopyran-4-yl)-1H-pyrazol-4-yloxy]-2,6-dimethyl-benzonitrile;
4-[3,5-dicyclopropyl-1-(tetrahydro-pyran-4-yl)-1H-pyrazol-4-yloxy]-2,6-dimethyl-benzonitrile;
4-[3,5-dicyclopropyl-1-(1,1-dioxo-tetrahydro-1λ⁶-thiophen-3-yl)-1H-pyrazol-4-yloxy]-2,6-dimethyl-benzonitrile;
4-(3-cyclopropyl-5-methyl-1-(1,1-dioxo-thietan-3-yl)-1H-pyrazol-4-yloxy)-2,6-dimethyl-benzonitrile;
4-(3-methyl-5-cyclopropyl-1-(1,1-dioxo-thietan-3-yl)-1H-pyrazol-4-yloxy)-2,6-dimethyl-benzonitrile;
4-[5-cyclobutyl-1-(1,1-dioxo-tetrahydro-1λ⁶-thiophen-3-yl)-3-methyl-1H-pyrazol-4-yloxy]-2,6-dimethyl-benzonitrile;
4-[3-cyclobutyl-1-(1,1-dioxo-tetrahydro-1λ⁶-thiophen-3-yl)-5-methyl-1H-pyrazol-4-yloxy]-2,6-dimethyl-benzonitrile;
4-[5-cyclopropyl-1-(1,1-dioxo-tetrahydro-1λ⁶-thiophen-3-yl)-3-methyl-1H-pyrazol-4-yloxy]-2,6-dimethyl-benzonitrile;
4-[3-cyclopropyl-1-(1,1-dioxo-tetrahydro-1λ⁶-thiophen-3-yl)-5-methyl-1H-pyrazol-4-yloxy]-2,6-dimethyl-benzonitrile;
4-(3-cyclopropyl-5-methyl-1-(1,1-dioxo-1λ⁶-thietan-3-yl)-1H-pyrazol-4-yloxy)-2-methyl-benzonitrile;
4-(3-cyclopropyl-5-methyl-1-(1,1-dioxo-1λ⁶-thietan-3-yl)-1H-pyrazol-4-yloxy)-2-chloro-benzonitrile;
4-[5-cyclopropyl-1-(1,1-dioxo-1λ⁶-thietan-3-yl)-1H-pyrazol-4-yloxy]-2,6-dimethyl-benzonitrile;
4-[3-cyclopropyl-1-(1,1-dioxo-1λ⁶-thietan-3-yl)-1H-pyrazol-4-yloxy]-2,6-dimethyl-benzonitrile;
4-[5-chloro-3-cyclopropyl-1-(1,1-dioxo-1λ⁶-thietan-3-yl)-1H-pyrazol-4-yloxy]-2,6-dimethyl-benzonitrile; and
4-[3-chloro-5-cyclopropyl-1-(1,1-dioxo-1λ⁶-thietan-3-yl)-1H-pyrazol-4-yloxy]-2,6-dimethyl-benzonitrile; or a salt, solvate, racemate, enantiomer, or prodrug thereof.

17. A pharmaceutical formulation comprising a compound, salt, solvate or prodrug according to claim 1 together with a pharmaceutically acceptable excipient, diluent or carrier.

18. A method of treatment of a mammal to treat endometriosis, uterine fibroids, menorrhagia, adenomyosis, primary and secondary dysmenorrhoea, or chronic pelvic pain syndrome comprising treating said mammal with an effective amount of a compound, salt, solvate or prodrug according to claim 1 or a composition thereof.

19. A method according to claim 18, wherein the disease or disorder is endometriosis or uterine fibroids.
